(19) United States
(12) Patent Application Publication
(10) Pub. No.: US 2016/0339008 A1
(43) Pub. Date: Nov. 24, 2016

(54) METHODS OF TREATING OR PREVENTING VASCULAR DISEASES OF THE RETINA

(71) Applicant: Children's Medical Center Corporation, Boston, MA (US)

(72) Inventors: Lois Smith, West Newton, MA (US); Zhuo Shao, Montreal (CA)

(21) Appl. No.: 15/031,631

(22) PCT Filed: Oct. 24, 2014
(86) PCT No.: PCT/US2014/062131

§ 371 Date: Apr. 22, 2016

Related U.S. Application Data
(60) Provisional application No. 61/895,851, filed on Oct. 25, 2013.

(51) Int. Cl. A61K 31/47; A61K 31/202; A61K 45/06; A61K 31/216

(52) U.S. Cl. CPC A61K 31/47; A61K 31/216; A61K 31/202; A61K 45/06

(57) ABSTRACT

The present invention features, in part, methods of treating or preventing vascular diseases of the retina in a subject, methods of treating or preventing angiogenesis in a subject and methods of treating or preventing neovascularization in a subject comprising administering to a subject a therapeutically effective amount of an inhibitor of cytochrome P450 2C8 (CYP2C8) activity or expression, or a promoter of sEH activity or expression.

### RELATED APPLICATIONS

The present application claims priority to, and the benefit under 35 U.S.C. §119(e) of U.S. provisional patent application No. 61/895,851, entitled “Methods of Treating or Preventing Vascular Diseases of the Retina,” filed Oct. 25, 2013. The entire content of the aforementioned patent application is incorporated herein by this reference.

### STATEMENT OF RIGHTS TO INVENTIONS MADE UNDER FEDERALLY SPONSORED RESEARCH

This work was supported by the following grant from the National Institutes of Health (NIH): 5RO1EY017017. The government has certain rights in the invention.

### BACKGROUND OF THE INVENTION

Vascular diseases of the retina, including diabetic retinopathy, exudative age related macular degeneration (ARMD), retinopathy of prematurity (ROP) and vascular occlusions, are major causes of visual impairment and blindness. This group of diseases is the focus of intense research aimed to identify novel treatment modalities that will help prevent or modify pathological ocular neovascularization. For example, ARMD affects millions of Americans over the age of 65 and causes visual loss in 10-15% of them as a direct effect of choroidal (sub-retinal) neovascularization. The leading cause of visual loss for Americans under the age of 65 is diabetes; millions of individuals in the United States are diabetic and many suffer from ocular complications of the disease, often a result of retinal neovascularization. While laser photocoagulation has been effective in preventing severe visual loss in subgroups of high risk diabetic patients, the overall 10-year incidence of retinopathy remains substantially unchanged. For patients with choroidal neovascularization due to ARMD or inflammatory eye disease such as ocular histoplasmosis, photocoagulation, with few exceptions, is ineffective in preventing visual loss.

Age related macular degeneration and diabetic retinopathy are the leading causes of visual loss in industrialized nations and do so as a result of abnormal retinal neovascularization. Since the retina consists of well-defined layers of neuronal, glial, and vascular elements, relatively small disturbances such as those seen in vascular proliferation or edema can lead to significant loss of visual function. Inherited retinal degenerations, such as retinitis pigmentosa (RP), are also associated with vascular abnormalities, such as arteriolar narrowing and vascular atrophy. While progress has been made in identifying factors that promote and inhibit angiogenesis, no treatment is currently available to specifically treat ocular vascular disease.

Inherited degenerations of the retina affect as many as 1 in 3500 individuals and are characterized by progressive night blindness, visual field loss, optic nerve atrophy, arteriolar attenuation, altered vascular permeability and central loss of vision often progressing to complete blindness. There are still no effective treatments to slow or reverse the progression of these retinal degenerative diseases.

Accordingly, there remains a need in the art for methods of treating or preventing vascular diseases of the retina, including retinopathy.

### SUMMARY OF THE INVENTION

Retinopathy with pathologic angiogenesis, a major cause of blindness, is suppressed with dietary ω3-polyunsaturated fatty acids (ω3PUFAs) through anti-angiogenic metabolites produced by cyclooxygenase (COX) and lipoxygenase (LOX). Additionally, cytochrome P450 (CYP) epoxygenases (CYP2C8), whose role in retinopathy remains unknown, metabolize PUFAs to produce epoxides, which are inactivated by soluble epoxide hydrolase (sEH) to form trans-dihydrodiols. The present invention is based, in part, on the novel finding that CYP2C8/sEH metabolism of ω3PUFA regulates neovascularization in oxygen-induced retinopathy (OIR), corresponding to an increased ω3PUFA epoxide:diol ratio. Inhibition of CYP2C8 presents a novel target for retinopathy treatment.

Accordingly, in a first aspect, the invention features a method of treating or preventing vascular diseases of the retina in a subject, comprising administering to a subject a therapeutically effective amount of an inhibitor of cytochrome P450 2C8 (CYP2C8) activity or expression, thereby treating or preventing vascular diseases of the retina.

In another aspect, the invention features a method of treating or preventing angiogenesis in a subject, comprising administering to a subject a therapeutically effective amount of an inhibitor of CYP2C8 activity or expression, thereby treating or preventing angiogenesis.

In still another aspect, the invention features a method of treating or preventing neovascularization in a subject, comprising administering to a subject a therapeutically effective amount of an inhibitor of CYP2C8 activity or expression, thereby treating or preventing neovascularization.

In a further aspect, the invention features a method of treating or preventing vascular diseases of the retina in a subject, comprising administering to a subject a therapeutically effective amount of a promoter of soluble epoxide hydrolase (sEH) activity or expression, thereby treating or preventing vascular diseases of the retina.

In an additional aspect, the invention provides a method for treating or preventing a vascular disease of the retina, angiogenesis and/or neovascularization in a subject that involves administering to a subject a therapeutically effective amount of montelukast or fenofibrate, such that treatment or prevention of a vascular disease of the retina, angiogenesis and/or neovascularization is achieved in the subject.

In one embodiment of the above aspects, the vascular diseases of the retina are selected from the group consisting of retinopathy, exudative age related macular degeneration (ARMD), and vascular occlusions. In a further embodiment, the retinopathy is selected from diabetic retinopathy and retinopathy of prematurity (ROP).

In another aspect, the invention features a method of treating or preventing angiogenesis in a subject, comprising administering to a subject a therapeutically effective amount of a promoter of sEH activity or expression, thereby treating or preventing angiogenesis.

In still another aspect, the invention features a method of treating or preventing neovascularization in a subject, comprising administering to a subject a therapeutically effective amount of a promoter of sEH activity or expression, thereby treating or preventing neovascularization.

In one embodiment of the above aspects, the subject is identified as having a vascular disease of the retina or as being predisposed to having a vascular disease of the retina. In a related embodiment, the vascular diseases of the retina are selected from the group consisting of: retinopathy, exudative age related macular degeneration (ARMD), and vascular occlusions.

In another embodiment of the above aspects, the subject is a prematurely delivered infant at risk for retinopathy of prematurity.

In one embodiment of the above aspects, montelukast, fenofibrate and/or the inhibitor of CYP2C8 decreases the activity of a CYP2C8 protein or decreases the expression of a CYP2C8 gene in the tissue. In another embodiment of the above aspects, the promoter of sEH increases the activity of a sEH protein or increases the expression of a sEH gene in the tissue. In a further embodiment, montelukast, fenofibrate, the inhibitor of CYP2C8 activity and/or promoter of sEH activity or expression is administered to ocular tissue.

In one embodiment of the above aspects, the retinopathy is selected from the group consisting of diabetic retinopathy, retinopathy of prematurity, and wet age-related macular degeneration.

In another further embodiment of the above aspects, the subject is being fed a polyunsaturated fatty acid (PUFA) enriched diet. In a related embodiment, the PUFA enriched diet is a ω3-PUFA diet or a ω-6 PUFA diet.

In an additional embodiment, a method of the invention further involves administration of an inhibitor of CYP2J2 to the subject. Optionally, the CYP2J2 inhibitor is Telmisartan, Flunarizine, Amodiaquine, Nicardipine, Mibefradil, Norfloxacin, Nifedipine, Nimodipine, Benzbromarone or Haloperidol.

Another aspect of the invention provides a pharmaceutical composition for treatment of a vascular disease of the retina in a subject that includes montelukast or fenofibrate and instructions for its use.

### DEFINITIONS

The following terms are provided solely to aid in the understanding of this invention. These definitions should not be construed to have a scope less than would be understood by a person of ordinary skill in the art.

In this disclosure, “comprises,” “comprising,” “containing” and “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

As used in this specification and the appended claims, the singular forms “a,” “an” and “the” include plural references unless the content clearly dictates otherwise.

The term “vascular diseases of the retina” as used herein is meant to refer to a range of eye diseases that affect the blood vessels in the eye. Exemplary vascular diseases of the retina include, but are not limited to, retinopathy, exudative age related macular degeneration (ARMD), and vascular occlusions.

The term “retinopathy” is meant to refer to persistent or acute damage to the retina of the eye. Types of retinopathy include diabetic retinopathy and retinopathy of prematurity (ROP).

The term “cytochrome P450” is meant to refer to a large and diverse group of enzymes that catalyze the oxidation of organic substances. Genes encoding CYP enzymes, and the enzymes themselves, are designated with the abbreviation CYP, followed by a number indicating the gene family, a capital letter indicating the subfamily, and another numeral for the individual gene. “Cytochrome P450 2C8 (CYP2C8)” is meant to refer to a member of the cytochrome P450 mixed-function oxidase system that is involved in the metabolism of xenobiotics in the body.

The term “angiogenesis” is meant to refer to the physiological process through which new blood vessels form from pre-existing vessels.

The term “neovascularization” is meant to refer to the development of tiny, abnormal, leaky blood vessels inside the eye.

The term “soluble epoxide hydrolase (sEH)” is meant to refer to a bifunctional enzyme that in humans is encoded by the EPHX2 gene. sEH is a member of the epoxide hydrolase family. This enzyme, found in both the cytosol and peroxisomes, binds to specific epoxides and converts them to the corresponding diols.

The term “polyunsaturated fat (PUFA)” is meant to refer to triglycerides in which the hydrocarbon tails constitutes polyunsaturated fatty acids (PUFA) (fatty acids possessing more than a single carbon-carbon double bond). ω3-PUFA refers to omega-3 fatty acids (also called ω-3 fatty acids or n-3 fatty acids) that are a group of three fats called ALA (found in plant oils), EPA, and DHA (both commonly found in marine oils).

The term “subject” as used herein includes animals, in particular humans as well as other mammals.

The term “treating” or “preventing” as used herein includes achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the subject, notwithstanding that the subject may still be afflicted with the underlying disorder. For prophylactic benefit, the compositions may be administered to a subject at risk of developing a particular disease, or to a subject reporting one or more of the physiological symptoms of a disease, even though a diagnosis of this disease may not have been made. The compositions may be administered to a subject to prevent progression of physiological symptoms or of the underlying disorder.

### ABBREVIATIONS AND ACRONYMS

PUFA—polyunsaturated fatty acids; COX—Cyclooxygenase; LOX—lipoxygenase; CYP—Cytochrome P450; sEH—soluble epoxide hydrolase; OIR—oxygen-induced retinopathy; DHA—docosahexaenoic acid; EPA—eicosapentaenoic acid; AA—arachidonic acid; EC—endothelial cells; VEGF—vascular endothelial growth factor; EET—epoxyeicosatrienoic acid; EDP—epoxydocosapentaenoic acids; EEQ—epoxyeicosatetraenoic acids; DHET—dihydroxy eicosatrienoic acid; DiHDPA—dihydroxy docosapentaenoic acid.

### BRIEF DESCRIPTION OF THE DRAWINGS

FIG. 1 shows retinal expression of CYP2C8 homologue, sEH and their products ratio in Normoxia versus OIR. (A) Schematic diagram of CYP2C8, and sEH metabolism of arachidonic acid (AA), docosahexaenoic acid (DHA) and eicosapentaenoic acid (EPA). (B) 3D reconstruction of confocal images of postnatal day (P) 17 normoxia and OIR retinal flat-mount stained with CYP2C (green), F4/80 (purple), isolectin (red) and DAPI (blue). Scale bar: 100 μm (C) Layer-by-layer confocal image across a vein of normoxia retina. (D) Co-localization of CYP2C and F4/80 (arrow) in OIR retinal flat-mount. (E) Retinal cross-sectional staining with isolectin (red), sEH (green) and DAPI (blue) show sEH is expressed in neovascular tufts (arrow head), as well as in neurons of the ganglion cell (GCL) and inner nuclear layers (INL). Scale bar: 10 μm (F) Blood smear indicates CYP2C-positive leukocytes (arrows). Scale bar: 20 μm (G) mRNA level of CYP2C in blood and retina with or without perfusion. (H) CYP2C and sEH mRNA expression in retina during OIR (n=6). (I) CYP2C and sEH protein expression in normoxia (N) versus OIR (0) retina (J) The ratio of corresponding AA, DHA and EPA epoxides to diols by LC/MS/MS oxylipid analysis (n=4-6/group) (two-way ANOVA with Bonferroni post-test, *p<0.05, **p<0.01, ***p<0.001).

FIG. 2 shows ω3PUFA feed modifies OIR neovascularization in Tie2-CYP2C8-Tg and Tie2-sEH-Tg mice. OIR neovascular area of: (A) Tie2-CYP2C8-Tg mice versus wild-type littermate control (WT) (n=11-13/group). (B) Tie2-sEH-Tg versus WT (n=14-19/group). (C) Systemic sEH knockout (sEH−/−) (n=8-15/group) Scale bar: 500 μm (D) RT-PCR of VEGF-A and VEGF-C in OIR Tie2-CYP2C8-Tg and Tie2-sEH-Tg versus WT (t-test, n.s.-not significant, *p<0.05, **p<0.01).

FIG. 3 shows Tie2-CYP2C8-Tg and Tie2-sEH-Tg alters the corresponding epoxides level in ω3PUFA-fed mice. Tie2-CYP2C8-Tg mice plasma levels of: (A) 14,15-EET, 19,20-EDP and 17,18-EEQ (n=4-6/group). (B) 14,15-EET, 19,20-EDP and 17,18-EEQ (n=4-6/group). Retinal ratios in Tie2-CYP2C8-Tg vs. WT of: (C) 14,15-EET:14,15-DHET, 19,20-EDP:DiHDPA and 17,18-EEQ:17,18-DHET (n=4-6/group). (D) Retinal ratios in Tie2-sEH-Tg vs. WT of 19,20-EDP:DiHDPA and 17,18-EEQ:17,18-DHET (n=4-6/group). (t-test, n.s.-not significant, *p<0.05, **p<0.01).

FIG. 4 shows aortic ring sprouting using Tie2-CYP2C8-Tg and Tie2-sEH-Tg treated with DHA and AA or epoxide metabolites. (A) AA (30 μM) or DHA (30 μM) induced aortic ring sprouting of WT and Tie2-CYP2C8-Tg mice (n=3-7/group). (B) Aortic sprouting from Tie2-sEH-Tg and sEH−/− treated with 17,18-EDP, 19,20-EEQ and 14,15-EET (n=4-8/group). Scale bars: 50 μm (t-test, n.s.-not significant, *p<0.05, **p<0.01)

FIG. 5 shows that with ω6PUFA-feed, Tie2-CYP2C8-Tg induces OIR-neovascularization versus WT (9.458±0.3425 vs. 8.291±0.3979, p=0.032); no difference was seen with Tie2-sEH-Tg or sEH−/−.

FIG. 6 shows that plasma 14,15-EET and retinal 14,15-EET:14,15-DHET ratio increased with Tie2-CYP2C8-Tg versus WT, consistent with increased neovascularization (A-D). With 14,15-EET treatment, aortic ring sprouting was similar in Tie2-sEH-Tg, sEH−/− and WT (E).

FIG. 7 shows that both low (10 mg/kg/day GV) and high (100 mg/kg/day GV) dose fenofibrate decreased neovascularization in JAX (WT) mice on normal feed.

FIG. 8 shows that both low (10 mg/kg/day GV) and high (100 mg/kg/day GV) dose fenofibrate decreased neovascularization in PPARα knockout mice on normal feed, indicating that the observed effect was PPARα-independent.

FIG. 9 shows that low dose fenofibrate decreased neovascularization in both WT and Cyp2C8 overexpressing transgenic (Tg) mice on both ω3 and ω6 LCPUFAfeed.

FIG. 10 shows that fenofibric acid (FA, active metabolite of fenofibrate) inhibited the sprouting of aortic rings from both WT & Cyp2C8 Tg mice. This inhibition was partially rescued by 19,20-EDP.

FIG. 11 shows that fenofibric acid (FA) suppressed the sprouting of aortic rings from both WT & Cyp2C8 Tg mice. This inhibition could not be rescued by DHA.

FIG. 12 shows that FA suppressed the sprouting of aortic rings from both WT & Cyp2C8 Tg mice, which could not be reversed by PPARalpha inhibitor GW6471.

FIG. 13 shows that was observed to inhibit human retinal microvascular endothelial cells (HRMEC) tubule formation, and this effect was partially rescued by 19,20 EDP.

FIG. 14 shows the results such as those shown in FIG. 13, quantitated and presented as histograms.

FIG. 15 shows that w3LCPUFA was unable to rescue the inhibition of HRMEC tubule formation by FA.

FIG. 16 shows the results such as those shown in FIG. 15, quantitated and presented as histograms.

FIG. 17 shows that fenofibrate was identified to inhibit HRMEC tubule formation in a manner that was PPARα-independent, when PPARα inhibitor GW6471 was examined and found to have no impact upon the observed effect of fenofibrate on HRMEC tubule formation.

FIG. 18 shows the results such as those shown in FIG. 17, quantitated and presented as histograms.

FIG. 19 shows that 19,20 EDP and 17,18 EEQ (a compound downstream of EPA and CYP2C8) were identified as partially rescuing the inhibition of HRMEC migration by FA.

FIG. 20 shows that w3LCPUFA was identified as incapable of rescuing HRMEC migration by FA

FIG. 21 shows that the FA inhibition of HRMEC migration was observed to be PPARα-independent, when PPARα inhibitor GW6471 was examined, and was found to have no impact upon the observed effect of fenofibrate on HRMEC migration.

FIG. 22 shows the assessed site of action of fenofibrate/FA within the ω3 and ω6 pathways.

FIG. 23 shows that Montelukast decreased neovascularization in JAX (WT) mice on normal feed.

FIG. 24 shows the impact of administering montelukast to mice overexpressing CYP2C8 (Cyp2C8 transgenic mice, “Cyp2C8 Tg”).

FIG. 25 shows the effects of montelukast on HRMEC tubule formation.

FIG. 26 shows that montelukast demonstrated clear dose-response curves when HRMEC tubule formation was assessed, with results also paralleling those observed for fenofibrate.

FIG. 27 demonstrates that HRMEC migration was inhibited by montelukast, in a manner that also showed a clear dose-response curve.

### DETAILED DESCRIPTION OF THE INVENTION

It has been previously demonstrated that a ω3PUFA-enriched diet in oxygen-induced retinopathy (OIR) suppresses neovascularization. The anti-angiogenic effects of ω3PUFAs in OIR pups are mainly derived from COX and LOX metabolites. Based on these studies, the addition of ω3PUFAs in the total parenteral nutrition for premature babies is in clinical trials to help prevent retinopathy. The newly identified, less characterized CYP pathway was recently found to metabolize ω6PUFA arachidonic acid (AA) to produce pro-angiogenic metabolites epoxyeicosatrienoic acids (EETs) but the role of ω3PUFA-derived CYP and sEH metabolites in retinopathy is unknown.

Understanding the role of CYP metabolites from ω3PUFA in retinopathy is critical to know the implications of adding ω3PUFAs to total parenteral nutrition. Described herein is a novel ω3PUFA epoxide metabolite from CYP2C8, which potentiates neovascularization. These results suggest that although COX and LOX ω3PUFA metabolites inhibits neovascularization in retinopathy, CYP2C8 ω3PUFA metabolites promote disease and the inhibition of CYP2C8 may provide a novel and interesting target for retinopathy treatment, as such inhibition would be expected to reduce or prevent production of pro-angiogenic metabolites from both ω3PUFA and ω6PUFA, both essential dietary fatty acids.

Cytochrome P450 (CYP) is a large and diverse superfamily of hemoproteins found in all domains of life. They use a plethora of both exogenous and endogenous compounds as substrates in enzymatic reactions. Usually they form part of multicomponent electron transfer chains, called P450-containing systems. Cytochrome P4502C8 (abbreviated CYP2C8), a member of the cytochrome P450 mixed-function oxidase system, is involved in the metabolism of xenobiotics in the body.

As described herein, the present invention includes inhibitors of cytochrome P4502C8 (CYP2C8) activity or expression. The present invention also includes activators, agonists and/or promoters of soluble epoxide hydrolase (sEH) activity or expression.

In certain embodiments, the inhibitor of CYP2C8 decreases the activity of a CYP2C8 protein or decreases the expression of a CYP2C8 gene in the cell or tissue. In other embodiments, the promoter of sEH increases the activity of a sEH protein or increases the expression of a sEH gene in the cell or tissue.

The present invention is not to be limited by type of inhibitor. Exemplary inhibitors of CYP2C8 or promoters of sEH include, but are not limited to, antibodies, peptides, inhibitory nucleic acids, such as siRNA, aptamers, and small organic molecules. “Small organic molecule” generally is used to refer to organic molecules of a size comparable to those organic molecules generally used in pharmaceuticals. The term typically excludes organic biopolymers (e.g., proteins, nucleic acids, etc.). Small organic molecules most often range in size up to about 5000 Da, in some embodiments, up to about 2000 Da, or in other embodiments, up to about 1000 Da. In certain embodiments, exemplary inhibitors of CYP2C8 activity or expression include fenofibrate, gemfibrozil, trimethoprim, thiazolidinediones, montelukast and quercetin. As examples, the chemical structures of fenofibrate and montelukast are

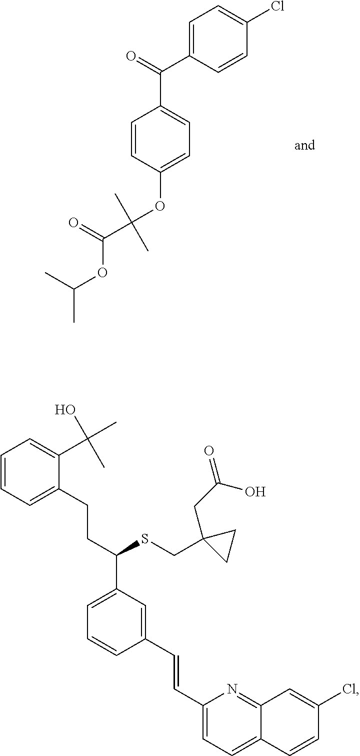 [SMILES: CC(C)OC(=O)C(C)(C)Oc1ccc(C(=O)c2ccc(Cl)cc2)cc1 ; CC(C)(O)c1ccccc1CC[C@@H](SCC1(CC(=O)O)CC1)c1cccc(/C=C/c2ccc3ccc(Cl)cc3n2)c1]

respectively. Additional exemplary inhibitors of CYP2C8 activity or expression include Candesartan cilexetil, Zafirlukast, Clotrimazole, Felodipine, Mometasone furoate, Salmeterol, Raloxifene, Ritonavir, Levothyroxine, Tamoxifen, Loratadine, Oxybutynin, Medroxyprogesterone, Simvastatin, Ketoconazole, Ethinyl estradiol, Spironolactone, Lovastatin, Nifedipine, Irbesartan, Clopidogrel, Amlodipine, Glyburide, Rosiglitazone, Cefuroxime axetil, Terfenadine, Pioglitazone, Dexamethazone, Rabeprazole, Tranylcypromine, Midazolam, Nystatin, Losartan, Paclitaxel, Exemestane, Valdecoxib, Fluvastatin, Celecoxib, Carvedilol, Triamcinolone, Estradiol, Nefazodone, Methylprednisolone, Sertraline and Candesartan (see Walsky et al. J. Clin. Pharmacol. 45: 68-78).

CYP2C8 or sEH activity or expression can be easily determined by one skilled in the art using routine assays, for example by immunohistochemical staining, enzyme-linked immunosorbent (ELISA) assay, western blot analysis, luminescent assays, mass spectrometry, high performance liquid chromatography, high-pressure liquid chromatography-tandem mass spectrometry and polymerase chain reaction (PCR) assays such as real time (RT) PCR. Fluorescence-based assays for screening cytochrome P450 (P450) activities in intact cells have been described (Donato et al. Drug Metab Dispos. 2004 July; 32(7):699-706; incorporated by reference in its entirety herein). Luminescent cytochrome p450 assays are commercially available from, e.g. PROMEGA.

In some embodiments, the inhibitor of CYP2C8 or the promoter of sEH is present in an amount sufficient to exert a therapeutic effect to reduce symptoms of a vascular disease of the retina by an average of at least about 5, 10, 15, 20, 25, 30, 40, 50, 60, 70, 80, 90, more than 90%, or substantially eliminate symptoms of the vascular disease of the retina.

In some embodiments, the inhibitor of CYP2C8 or the promoter of sEH is present in an amount sufficient to exert a therapeutic effect to reduce symptoms of retinopathy, for example diabetic retinopathy, by an average of at least about 5, 10, 15, 20, 25, 30, 40, 50, 60, 70, 80, 90, more than 90%, or substantially eliminate symptoms of retinopathy.

In other embodiments, the inhibitor of CYP2C8 or the promoter of sEH is present in an amount sufficient to reduce retinal degeneration in a subject by an average of at least about 5, 10, 15, 20, 25, 30, 40, 50, 60, 70, 80, 90, more than 90%, or substantially eliminate retinal degeneration.

In some embodiments, the inhibitor of CYP2C8 or the promoter of sEH is present in an amount sufficient to decrease vascular occlusions in a treated eye of a subject by an average of at least about 5, 10, 15, 20, 25, 30, 40, 50, 60, 70, 80, 90, more than 90%, or substantially eliminate retinal edema.

In yet other embodiments, the inhibitor of CYP2C8 or the promoter of sEH is present in an amount sufficient to decrease angiogenesis in a treated eye of a subject by an average of at least about 5, 10, 15, 20, 25, 30, 40, 50, 60, 70, 80, 90, more than 90%, or substantially eliminate angiogenesis.

In some embodiments, the inhibitor of CYP2C8 or the promoter of sEH is present in an amount sufficient to decrease retinal neovascularization in a treated eye of a subject by an average of at least about 5, 10, 15, 20, 25, 30, 40, 50, 60, 70, 80, 90, more than 90%, or substantially eliminate retinal neovascularization.

In some embodiments, the inhibitor of CYP2C8 or the promoter of sEH is present in an amount sufficient to retard loss of vision in a treated eye of a subject by an average of at least about 5, 10, 15, 20, 25, 30, 40, 50, 60, 70, 80, 90, more than 90%, or substantially eliminate further loss of vision.

In some embodiments, the inhibitor of CYP2C8 or the promoter of sEH is present in an amount sufficient to limit non-proliferative damage to a retina of a subject by an average of at least about 5, 10, 15, 20, 25, 30, 40, 50, 60, 70, 80, 90, more than 90%, or substantially eliminate the non-proliferative damage to the retina.

In some embodiments, the inhibitor of CYP2C8 or the promoter of sEH is present in an amount sufficient to slow proliferative damage to a retina of a subject by an average of at least about 5, 10, 15, 20, 25, 30, 40, 50, 60, 70, 80, 90, more than 90%, or substantially eliminate further proliferative damage to the retina.

The compounds of the invention may be obtained commercially, or prepared by methods well known to those skilled in the art, or disclosed in the references incorporated herein and may be purified in a number of ways, including by crystallization or precipitation under varied conditions to yield one or more polymorphs.

### Methods of Treatment

Included in the present invention are methods of treating or preventing vascular diseases of the retina in a subject, methods of treating or preventing angiogenesis in a subject, and methods of treating or preventing neovascularization in a subject, comprising administering to a subject a therapeutically effective amount of an inhibitor of cytochrome P450 2C8 (CYP2C8) activity or expression. Also included in the invention are methods of treating or preventing vascular diseases of the retina in a subject, comprising administering to a subject a therapeutically effective amount of a promoter of soluble epoxide hydrolase (sEH) activity or expression.

The term “subject” as used herein includes animals, in particular humans as well as other mammals. In certain embodiments, the subject is a prematurely delivered infant at risk for retinopathy of prematurity. In other embodiments, the subject is suffering from diabetes. In other embodiments, the subject is identified as being predisposed to having vascular diseases of the retina.

In certain embodiments, the invention features a method of treating or preventing vascular diseases of the retina in a subject, comprising administering to a subject a therapeutically effective amount of an inhibitor of cytochrome P450 2C8 (CYP2C8) activity or expression, or a therapeutically effective amount of a promoter of soluble epoxide hydrolase (sEH) activity or expression, thereby treating or preventing retinopathy.

In other embodiments, the invention features a method of treating or preventing angiogenesis in a subject, comprising administering to a subject a therapeutically effective amount of an inhibitor of CYP2C8 activity or expression, or a therapeutically effective amount of a promoter of soluble epoxide hydrolase (sEH) activity or expression, thereby treating or preventing angiogenesis.

In still other embodiments, the invention also features a method of treating or preventing neovascularization in a subject, comprising administering to a subject a therapeutically effective amount of an inhibitor of CYP2C8 activity or expression, or therapeutically effective amount of a promoter of soluble epoxide hydrolase (sEH) activity or expression, thereby treating or preventing neovascularization.

Conditions and diseases amenable to prophylaxis or treatment with inhibitors of cytochrome P450 2C8 (CYP2C8) activity or expression invention include but are not limited to those in which abnormal vascular or cellular proliferation occurs. In certain embodiments, the disease or condition is wherein a vascular disease of the retina. For example, vascular diseases of the retina can be retinopathy, exudative age related macular degeneration (ARMD), and vascular occlusions. Retinopathy is due to persistent or acute damage to the retina of the eye. Ongoing inflammation and vascular remodeling may occur over periods of time where the patient is not fully aware of the extent of the disease. Frequently, retinopathy is an ocular manifestation of systemic disease as seen in diabetes or hypertension. In particular embodiments, the retinopathy is selected from diabetic retinopathy and retinopathy of prematurity (ROP).

Retinopathy of prematurity (ROP) occurs in premature neonates. Normally, the retina becomes completely vascularized at full term. In the premature baby, the retina remains incompletely vascularized at the time of birth. Rather than continuing in a normal fashion, vasculogenesis in the premature neonatal retina becomes disrupted. Abnormal new proliferating vessels develop at the juncture of vascularized and avascular retina. These abnormal new vessels grow from the retina into the vitreous, resulting in hemorrhage and tractional detachment of the retina. Although laser ablation of avascular peripheral retina may halt the neovascular process if delivered in a timely and sufficient manner, some premature babies nevertheless go on to develop retinal detachment. Surgical methods for treating ROP-related retinal detachments in neonates have limited success at this time because of unique problems associated with this surgery, such as the small size of the eyes and the extremely firm vitreoretinal attachments in neonates.

Diabetic retinopathy is the leading cause of blindness in adults of working age. In persons with diabetes mellitus, retinal capillary occlusions develop, creating areas of ischemic retina. Retinal ischemia serves as a stimulus for neovascular proliferations that originate from pre-existing retinal venules at the optic disk or elsewhere in the retina posterior to the equator. Severe visual loss in proliferative diabetic retinopathy (PDR) results from vitreous hemorrhage and tractional retinal detachment. Again, laser treatment (pan retinal photocoagulation to ischemic retina) may arrest the progression of neovascular proliferations in this disease but only if delivered in a timely and sufficiently intense manner. Some diabetic patients, either from lack of ophthalmic care or despite adequate laser treatment, go on to sustain severe visual loss secondary to PDR. Vitrectomy surgery can reduce but not eliminate severe visual loss in this disease.

Age-related macular degeneration is the leading cause of severe visual loss in persons over 65 years old. In contrast to ROP and PDR, in which neovascularization emanates from the retinal vasculature and extends into the vitreous cavity, AMD is associated with neovascularization originating from the choroidal vasculature and extending into the subretinal space. Choroidal neovascularization causes severe visual loss in AMD patients because it occurs in the macula, the area of retina responsible for central vision. The stimuli which lead to choroidal neovascularization are not understood. Laser ablation of the choroidal neovascularization may stabilize vision in selected patients. However, only 10% to 15% of patients with neovascular AMD have lesions judged to be appropriate for laser photocoagulation according to current criteria.

Retinopathy of prematurity, proliferative diabetic retinopathy, and neovascular age-related macular degeneration are but three of the ocular diseases which can produce visual loss secondary to neovascularization. Others include sickle cell retinopathy, retinal vein occlusion, and certain inflammatory diseases of the eye. These, however, account for a much smaller proportion of visual loss caused by ocular neovascularization.

Retinopathy is modeled in the mouse eye with oxygen-induced vessel loss, which precipitates hypoxia-induced retinopathy, allowing for assessment of retinal vessel loss, vessel regrowth after injury and pathological angiogenesis.

Non-proliferative diabetic retinopathy (NPDR) demonstrates, at its outset, abnormalities of the normal microvascular architecture characterized by degeneration of retinal capillaries, formation of saccular capillary microaneurysms, pericyte deficient capillaries, and capillary occlusion and obliteration. Mechanisms of action include diabetes-induced vascular inflammation leading to occlusion of the vascular lumen by leukocytes and platelets followed by the eventual death of both pericytes and endothelial cells. Attraction and adhesion of leukocytes to the vascular wall by the inflammatory process cause leukocytes to adhere temporarily to the endothelium (leukostasis), release cytotoxic factors, and injure or kill the endothelial cell. The damaged endothelial surface initiates platelet adherence, aggregation, microthrombi formation, vascular occlusion and ischemia. Another consequence of endothelial injury is alteration in the Blood-Retinal Barrier (BRB) causing increased vascular permeability. This can be evidenced by fluorescein leakage during fluorescein angiography or retinal thickening assessed by optical coherence tomography (OCT). Consequences of this leakage can be clinically significant macular edema and deposition of lipoproteins in the retina (hard exudates) contributing to retinal thickening. As the process continues, retinal ganglion cells are lost leading towards visual loss or blindness. The disrupted autoregulation and decreased retinal blood flow resulting from the changes in vasculature in endothelial cells, pericyte death, and capillary obliteration are markers for progression of DR, and leads to development of retinal ischemia, which enables development of the more severe, proliferative stage of DR.

Proliferative DR involves neovascularization or angiogenesis, induced by retinal ischemia of the disc or other locations of the retina. This new vasculature can cause hemorrhage of the vitreous humour and retinal detachments from accompanying contractile fibrous tissue.

At any point during this progression of diabetic retinopathy, macular edema or diabetic macular edema (DME) can develop, with severe impact on vision function. Progression of this associated disorder is predicted by retinal vascular leakage and leads to photocoagulation treatment in order to reduce the risk of vision loss. Since a large proportion of patients with diabetic retinopathy suffer from this disorder as well, it is a relevant clinical intervention target. All of these injuries or degenerative insults may lead to impairment or even complete loss of visual acuity and offer targets for therapeutic intervention. No efficient therapeutic options currently are available. Laser photocoagulation involves administering laser burns to various areas of the eye and is used in the treatment of many neovascularization-linked disorders. Neovascularization, in particular, is commonly treated with scatter or panretinal photocoagulation. However, laser treatment may cause permanent blind spots corresponding to the treated areas. Laser treatment may also cause persistent or recurrent hemorrhage, increase the risk of retinal detachment, or induce neovascularization or fibrosis. Other treatment options for ocular-related disorders include thermotherapy, vitrectomy, photodynamic therapy, radiation therapy, surgery, e.g., removal of excess ocular tissue, and the like. However, in most cases, all available treatment options have limited therapeutic effect, require repeated, costly procedures, and/or are associated with dangerous side-effects.

Many types of retinopathy are proliferative, resulting, most often, from neovascularization or the overgrowth of blood vessels. Angiogenesis may result in blindness or severe vision loss, particularly if the macula becomes affected. In some rare cases, retinopathy can be due to genetic diseases such as retinitis pigmentosa. In other therapeutic interventions which can be associated with diabetic complications in the eye, vitrectomy procedures may be utilized. Dexamethasone, a glucocorticoid steroid, has been shown to be useful in reducing post-operative inflammation which can be enhanced in diabetic subjects relative to non-diabetic subjects. Thus, it may be desirable to perform the methods of the invention in combination with dexamethasone.

Combination therapies involving, e.g., administration of an inhibitor of CYP2C8 (e.g., montelukast, fenofibrate or other) with an inhibitor of CYP2J2 are also contemplated. Exemplary inhibitors of CYP2J2 include Telmisartan, Flunarizine, Amodiaquine, Nicardipine, Mibefradil, Norfloxacin, Nifedipine, Nimodipine, Benzbromarone, Haloperidol, Metoprolol, Triamcinolone, Perphenazine, Bepridil, Clozapine, Sertraline, Ticlopidine, Verapamil, Chlorpromazine and Ceftriaxone (see Ren et al. Drug Metab. Dispos. 41: 60-71).

In other therapeutic interventions which can be associated with diabetic complications in the eye, photodynamic therapy may be utilized to correct occlusion or leakiness, and may cause excessive inflammation in a diabetic subject. Laser photocoagulation therapy may be utilized to correct occlusion or leakiness, and may cause excessive inflammation in a diabetic subject. Thus, it may be desirable to use a therapeutically effective amount of an inhibitor of cytochrome P450 2C8 (CYP2C8) activity or expression in combination with photodynamic therapy. A therapeutically effective amount of an inhibitor of cytochrome P450 2C8 (CYP2C8) activity or expression of the invention may be administered to a subject prior to the therapy.

Individuals with DME have higher risk of cataract development which is a frequent cause of vision loss. Diabetic patients have a higher risk of both anterior and posterior segment complications following cataract surgery. One of the most significant of these is neovascularization of the iris as it can progress to neovascular glaucoma. Other anterior chamber complications include pigment dispersion with precipitates on the surface of the newly implanted intraocular lens (IOL), fibrinous exudates or membrane formation (from inflammation) in the anterior chamber. In some embodiments of the invention, reduction in anterior or posterior segment complications following cataract surgery in an eye of a subject with DME can be achieved by administering an inhibitor of cytochrome P450 2C8 (CYP2C8) activity or expression to a subject in need thereof. In some embodiments, methods are provided to prophylactically administer an inhibitor of cytochrome P450 2C8 (CYP2C8) activity or expression to a subject with DME who is at higher risk of developing cataracts compared to a healthy subject, thereby reducing or preventing developing cataracts.

Other such conditions and diseases that may be treated by the methods of the invention, e.g. by administering to a subject a therapeutically effective amount of an inhibitor of cytochrome P450 2C8 (CYP2C8) activity or expression, include diseases characterized by angiogenesis or neovascularization. For example, proliferative diseases including cancer and psoriasis, various inflammatory diseases characterized by proliferation of cells such as atherosclerosis and rheumatoid arthritis, where suppression of cellular proliferation is a desired goal in the treatment of these and other conditions. In certain examples, preventing both angiogeneisis and proliferation may be beneficial in the treatment of, for example, solid tumors, in which both the dysproliferative cells and the enhanced tumor vasculature elicited thereby are targets for inhibition by the agents of the invention. In either case, therapy to promote or suppress proliferation may be beneficial locally but not systemically, and for a particular duration, and proliferation-modulating therapies should be appropriately applied. The invention embraces localized delivery of such compounds to the affected tissues and organs, to achieve a particular effect.

Non-limiting examples of cancers, tumors, malignancies, neoplasms, and other dysproliferative diseases that can be treated according to the invention include leukemias such as myeloid and lymphocytic leukemias, lymphomas, myeloproliferative diseases, and solid tumors, such as but not limited to sarcomas and carcinomas such as fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, prostate cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms' tumor, cervical cancer, testicular tumor, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodendroglioma, meningioma, melanoma, neuroblastoma, and retinoblastoma.

As mentioned above, vascularization of the vitreous humor of the eye as a consequence of diabetic retinopathy is a major cause of blindness, and inhibition of such vascularization is desirable. Other conditions in which vascularization is undesirable include certain chronic inflammatory diseases, in particular inflammatory joint and skin disease, but also other inflammatory diseases in which a proliferative response occurs and is responsible for part or all of the pathology. For example, psoriasis is a common inflammatory skin disease characterized by prominent epidermal hyperplasia and neovascularization in the dermal papillae. Proliferation of smooth muscle cells, perhaps as a consequence of growth factors, is a factor in the narrowing and occlusion of the macrovasculature in atherosclerosis, responsible for myocardial ischemia, angina, myocardial infarction, and stroke, to name a few examples. Peripheral vascular disease and arteriosclerosis obliterans comprise an inflammatory component.

In some embodiments of the invention, the subject is fed a polyunsaturated fatty acid (PUFA) enriched diet, and in particular a ω3-PUFA enriched diet. Polyunsaturated fatty acids (PUFAs) are fatty acids that contain more than one double bond in their backbone. Polyunsaturated fatty acids can be classified in various groups by their chemical structure: omega-3, omega-6 and omega-9. Exemplary omega-3 fatty acids include, but are not limited to, Hexadecatrienoic acid (HTA), Alpha-linolenic acid (ALA), Stearidonic acid (SDA), Eicosatrienoic acid (ETE), Eicosatetraenoic acid (ETA), Eicosapentaenoic acid (EPA, Timnodonic acid), Heneicosapentaenoic acid (HPA), Docosapentaenoic acid (DPA, Clupanodonic acid), Docosahexaenoic acid (DHA, Cervonic acid), Tetracosapentaenoic acid and Tetracosahexaenoic acid (Nisinic acid). Exemplary omega-6 fatty acids include, but are not limited to, Linoleic acid, Gamma-linolenic acid (GLA), Eicosadienoic acid, Dihomo-gamma-linolenic acid (DGLA), Arachidonic acid (AA), Docosadienoic acid, Adrenic acid, Docosapentaenoic acid (Osbond acid), Tetracosatetraenoic acid and Tetracosapentaenoic acid. Exemplary omega-9 fatty acids include, but are not limited to, Oleic acid, Eicosenoic acid, Mead acid, Erucic acid and Nervonic acid.

In some embodiments of the invention a diagnostic test is included in a method of treatment with a therapeutically effective amount of an inhibitor of cytochrome P450 2C8 (CYP2C8) activity or expression or a promoter of soluble epoxide hydrolase (sEH) activity or expression. In one embodiment, a diagnostic test for diabetic retinopathy is performed and after a diagnosis of the disease is made, the subject is administered an inhibitor of cytochrome P450 2C8 (CYP2C8) activity or expression or a promoter of soluble epoxide hydrolase (sEH) activity or expression as described herein. In some embodiments of the invention, the diagnostic test is performed by imaging an eye of the subject or analysis of a biological sample of an eye of the subject.

### Administration

In some of the embodiments of the invention, the therapeutic agent, e.g. a therapeutically effective amount of an inhibitor of cytochrome P450 2C8 (CYP2C8) activity or expression or a therapeutically effective amount of a promoter of sEH activity or expression, is administered topically, orally, periocularly, intraocularly, via injection, nasally, via an aerosol, via an insert, via an implanted device, or via a drop. In other of the embodiments of the invention, the therapeutic agent is administered in a carrier vehicle which is liquid drops, liquid wash, nebulized liquid, gel, ointment, aerosol, spray, polymer micro and nanoparticles, solution, suspension, solid, biodegradable matrix, powder, crystals, foam, or liposomes. In some of the embodiments of the invention, a therapeutically effective amount of said therapeutic agent is delivered to an eye of said subject via local or systemic delivery. In some of the embodiments of the invention an injectable administration is performed intraocularly or periocularly. In some embodiments of the invention, administration is accomplished by administering an intra-ocular instillation of a gel, cream, powder, foam, crystals, liposomes, spray, polymer micro or nanospheres, or liquid suspension form of said compound. In some of the embodiments, polymer micro or nanospheres are used to deliver the therapeutic agent via periocular or intraocular injection or implantation.

In some of the embodiments, a therapeutically effective amount of the therapeutic agent is delivered to an eye of the subject via local or systemic delivery.

In some of the embodiments of the invention, the therapeutic agent is administered in a carrier vehicle which is liquid drops, liquid wash, nebulized liquid, gel, ointment, aerosol, spray, polymer micro and nanoparticles, solution, suspension, solid, biodegradable matrix, powder, crystals, foam, or liposomes. In some of the embodiments of the invention, topical administration comprises infusion of said compound to said eyes via a device selected from the group consisting of a pump-catheter system, an insert, a continuous or selective release device, a bioabsorbable implant, a continuous or sustained release formulation, and a contact lens. In some of the embodiments of the invention, injectable administration is performed intraocularly, intravitreally, periocularly, subcutaneously, subconjunctivally, retrobulbarly, or intracamerally. Controlled release formulations are also provided for in some embodiments of the invention. In some embodiments of the invention, the compounds of the invention are formulated as prodrugs. In some embodiments of the invention the formulation of the therapeutic agent includes no preservative. In some embodiments of the invention the formulation of the therapeutic agent includes at least one preservative. In some embodiments of the invention the formulation of the therapeutic agent includes a thickening agent. In other embodiments of the invention, the formulation of the therapeutic agent uses micro- or nanoparticles.

The compound is administered to the subject in an amount sufficient to achieve intraocular or retinal concentrations determined by a skilled clinician to be effective, for example in an amount sufficient to achieve intraocular or retinal concentrations of from about 1×10⁻⁸ to about 1×10⁻¹ moles/liter. In some embodiments of the invention, the compound is administered at least once a year. In other embodiments of the invention, the compound is administered at least once a day. In other embodiments of the invention, the compound is administered at least once a week. In some embodiments of the invention, the compound is administered at least once a month.

Exemplary doses for administration of a CYP2C8 and/or other CYP inhibitor to a subject include, but are not limited to, the following: 1-20 mg/kg/day, 2-15 mg/kg/day, 5-12 mg/kg/day, 10 mg/kg/day, 1-500 mg/kg/day, 2-250 mg/kg/day, 5-150 mg/kg/day, 20-125 mg/kg/day, 50-120 mg/kg/day, 100 mg/kg/day, at least 10 ug/kg/day, at least 100 ug/kg/day, at least 250 ug/kg/day, at least 500 ug/kg/day, at least 1 mg/kg/day, at least 2 mg/kg/day, at least 5 mg/kg/day, at least 10 mg/kg/day, at least 20 mg/kg/day, at least 50 mg/kg/day, at least 75 mg/kg/day, at least 100 mg/kg/day, at least 200 mg/kg/day, at least 500 mg/kg/day, at least 1 g/kg/day, and a therapeutically effective dose that is less than 500 mg/kg/day, less than 200 mg/kg/day, less than 100 mg/kg/day, less than 50 mg/kg/day, less than 20 mg/kg/day, less than 10 mg/kg/day, less than 5 mg/kg/day, less than 2 mg/kg/day, less than 1 mg/kg/day, less than 500 ug/kg/day, and less than 500 ug/kg/day.

In some embodiments of the invention, a second therapeutic agent is administered prior to, in combination with, at the same time, or after administration of the therapeutically effective amount of an inhibitor of cytochrome P450 2C8 (CYP2C8) activity or expression or a therapeutically effective amount of a promoter of sEH activity or expression. In some embodiments, the second therapeutic agent is selected from the group consisting of antioxidants, antiinflammatory agents, antimicrobials, steroids, protein kinase C inhibitors, angiotensin converting enzyme inhibitors, antiangiogenic agents, complement inhibitors, a CYP 2J2 inhibitor and anti-apoptotic agents. In some embodiments of the invention, the second therapeutic agent is an antibody or antibody fragment.

The representative examples that follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that the contents of those cited references are incorporated herein by reference to help illustrate the state of the art.

### Examples

Described herein is a novel ω3PUFA metabolite from CYP2C8, which potentiates neovascularization. These results indicated that although a ω3PUFA-enriched diet overall inhibits neovascularization in retinopathy, the inhibition of CYP2C8 could provide a new and attractive target for retinopathy treatment, as blocking CYP2C8 could inhibit production of pro-angiogenic metabolites from both ω3PUFA and ω6PUFA, both essential dietary fatty acids.

The results described herein demonstrate, in part, a pro-angiogenic role of a ω3PUFA metabolite 14,15-EDP from CYP2C8 and an anti-angiogenic role of soluble epoxide hydrolase (sEH), mainly achieved by increasing the breakdown of 14,15-EDP through this epoxygenase pathway, as has been demonstrated for the first time herein. The results described herein demonstrate the importance of considering both the production and breakdown of active metabolites to influence angiogenesis in retinopathy. The results described herein also demonstrate that CYP2C8 produces a pro-angiogenic pro-retinopathy metabolite from both ω6PUFA (14,15-EET) and from ω3PUFA (14,15-EDP), which presents an interesting therapeutic target for retinopathy treatment—inhibition of CYP2C8. Further, the results described herein show that in retina, the CYP2C8 positive cells and metabolites come from the circulation, causing the increased level of the pro-angiogenic 14,15-EDP (and 14,15-EET). The leukocyte source of CYP2C8 has never been shown before.

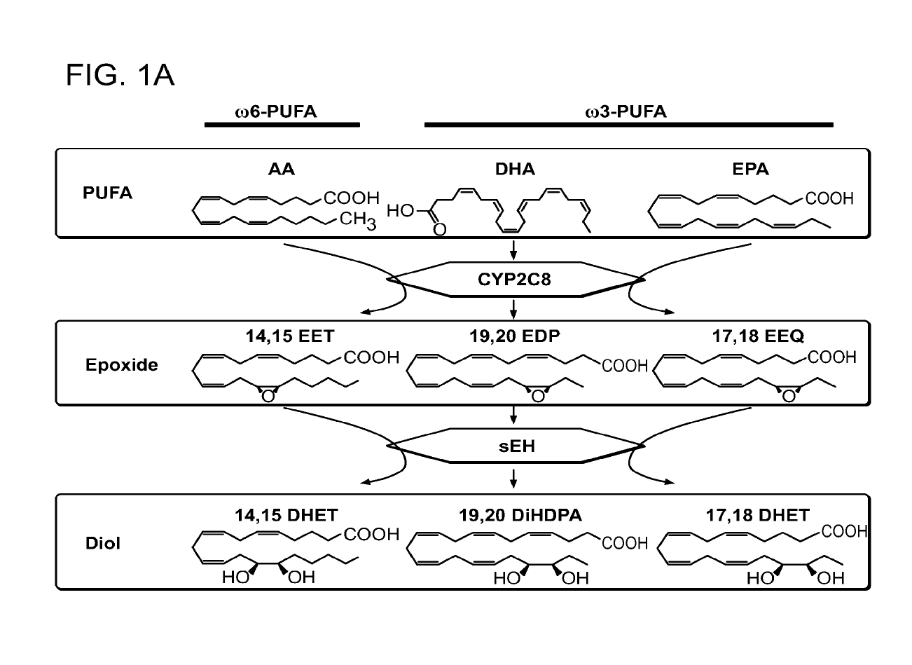

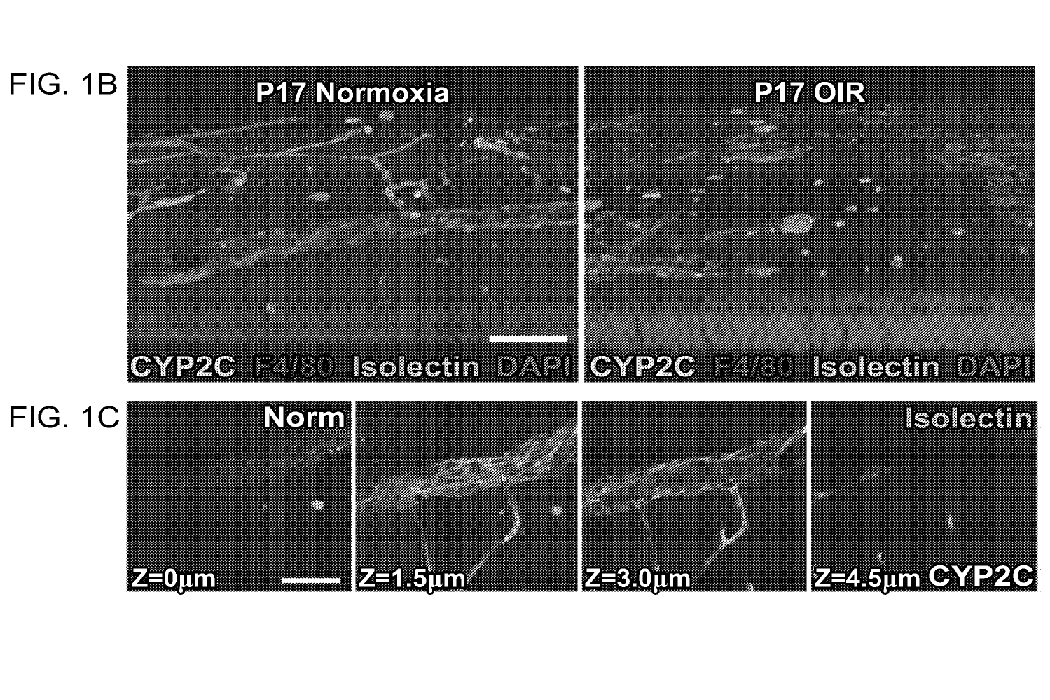

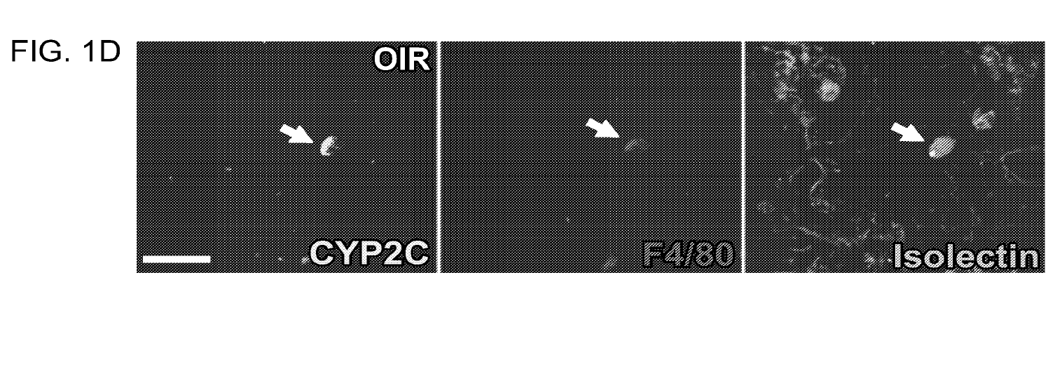

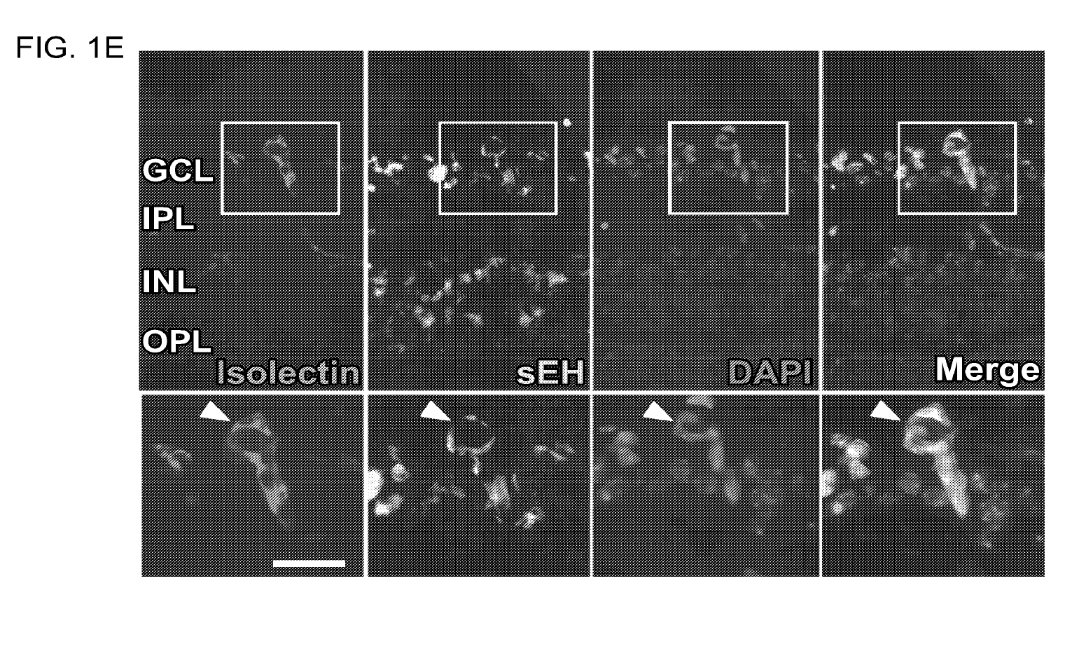

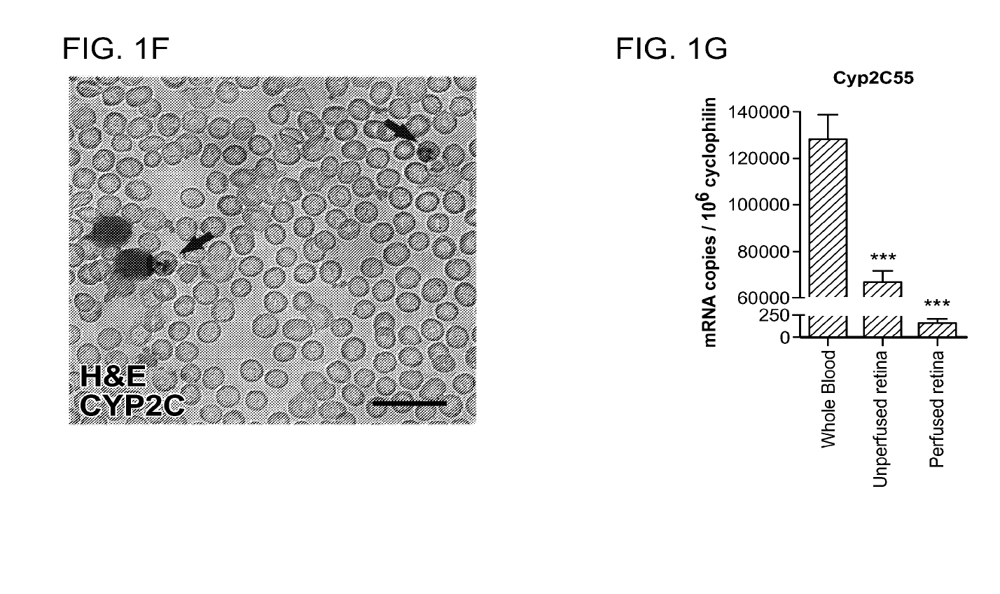

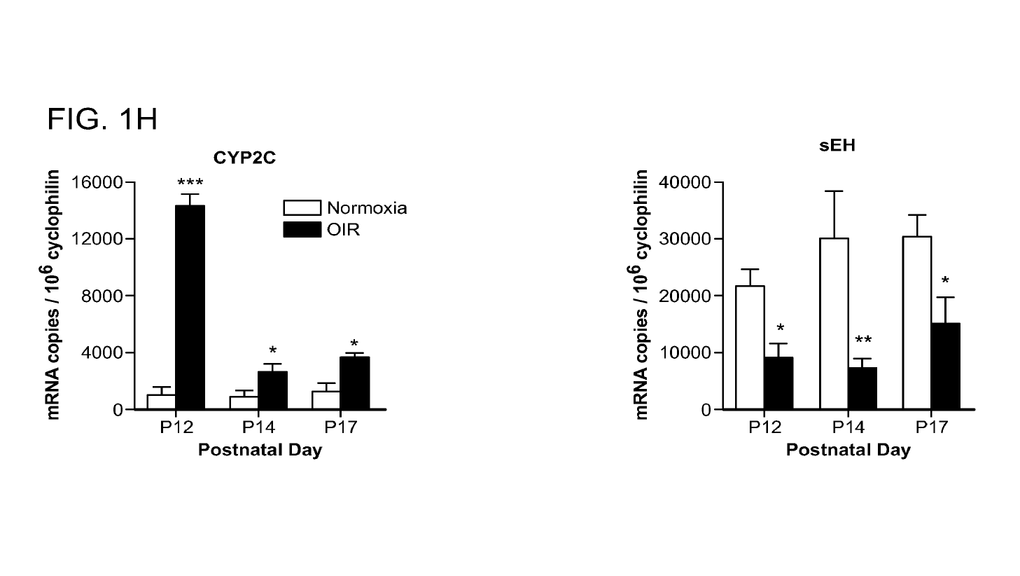

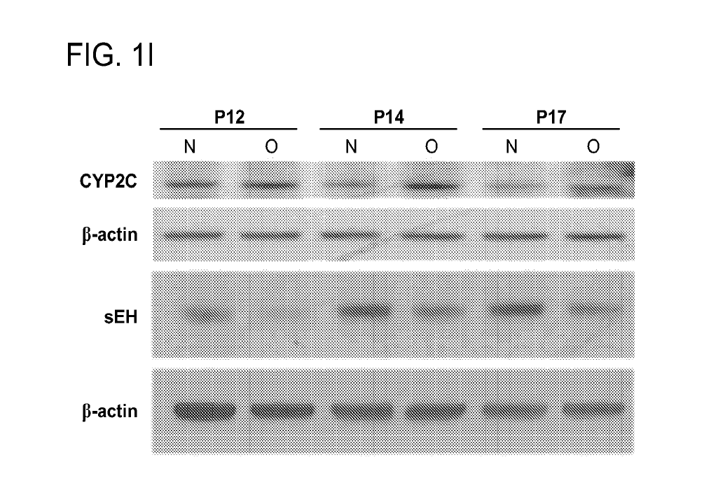

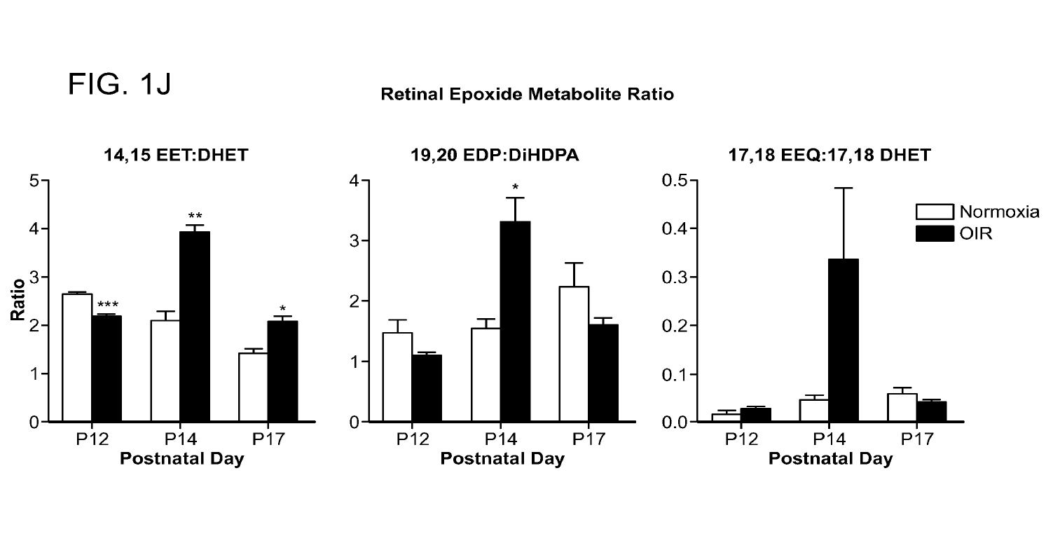

Pathologic neovascularization in retinopathy is a major cause of blindness. Finding effective treatment is critical. Omega-3 polyunsaturated fatty acids (w3PUFA), docosahexaenoic acid (DHA) and eicosapentaenoic acid (EPA), protect against the development of retinopathy in animal and clinical studies^{1,2} through active metabolites of cyclooxygenase (COX) and lipoxygenase (LOX)^{2,3}. Cytochrome P450s (CYPs) also metabolize both ω3PUFAs and ω6PUFAs into epoxides, which are further hydrolyzed by soluble epoxide hydrolase (sEH) to form less active trans-dihydrodiols (diols), hence dampening the biological effects of PUFA epoxides. (FIG. 1A)^{4,5}. It is therefore important to elucidate the role of both enzymes (CYP2C) that generates active metabolites and enzymes (sEH) that break them down, and decipher their impact on retinopathy.

CYP2C8, a dominant epoxygenase in humans, is induced by hypoxia⁶, a critical factor in retinopathy development. SEH is implicated in cardiovascular diseases⁸, and expressed in ECs⁷ and hence may directly regulate angiogenesis.

Although ω6PUFA-derived epoxyeicosatrienoic acids (EETs) synthesized by CYP2C8 from arachidonic acid (AA), promote angiogenesis¹⁰, the angiogenic effect on retinopathy of ω3PUFA-derived epoxy metabolites from CYP2C8: DHA-derived epoxydocosapentaenoic acids (EDPs), and EPA-derived epoxyeicosatetraenoic acids (EEQs) is unknown. However, both exhibit potent vasodilatory and cardio-protective effects¹¹, with EDPs suggested to suppress EC migration and angiogenesis in tumors¹².

In the experiments described herein, the role of CYP2C8 and its ω3PUFA metabolites in OIR were investigated using endothelial cells (EC) and monocyte/macrophage-specific CYP2C8 and sEH overexpressing mice (Tie2-CYP2C8-Tg, Tie2-sEH-Tg), as well as germ-line knockout of sEH (sEH−/−) and their WT littermate controls with a ω3PUFA-enriched diet. CYP2C8 and sEH metabolites from ω6PUFA were similarly examined in OIR.

### Example 1

### Expression of CYP2C, sEH and their Metabolites in OIR Versus Normoxia

Mouse CYP2C8 homologue (CYP2C)-positive cells have been found within blood vessel lumens in normoxic retinas (FIGS. 1B&C) and outside vessels in P17 OIR retinas, consistent with monocyte/macrophage migration from leaky vessels (FIG. 1B). F4/80-positive macrophages also have been identified to express CYP2C in OIR (FIG. 1D). Pathologic neovessels and neural tissue have been identified to express sEH in OIR (FIG. 1E). CYP2C-positive leukocytes have been detected in blood cells from WT normoxia mice (FIG. 1F). The mRNA level of CYP2C has been identified as highest in whole blood and dramatically higher in non-perfused versus perfused retina, indicating that CYP2C in normal retina originates from blood cells (FIG. 1G).

CYP2C was confined to be induced in retina (both mRNA and protein) during OIR, whereas sEH was suppressed (p<0.05; FIGS. 1H&I). The recruitment of CYP2C-expressing macrophages accompanied by increased vascular leakage may have contribute to the increased CYP2C in OIR retinas. In OIR versus normoxia at P14 (on normal chow), the retinal epoxide:diol ratio of AA to DHA was increased >2-fold (14,15-EET:14,15-DHET (p=0.0073) and 19,20-EDP: 19,20DiHDPA (p=0.017)) (FIG. 1J), consistent with increased CYP2C and decreased sEH levels.

### Example 2

### Impact of ω3PUFA Feed on Retinopathy with Tie2-CYP2C8-Tg, Tie2-sEH-Tg and sEH−/− Mice and VEGF Expression

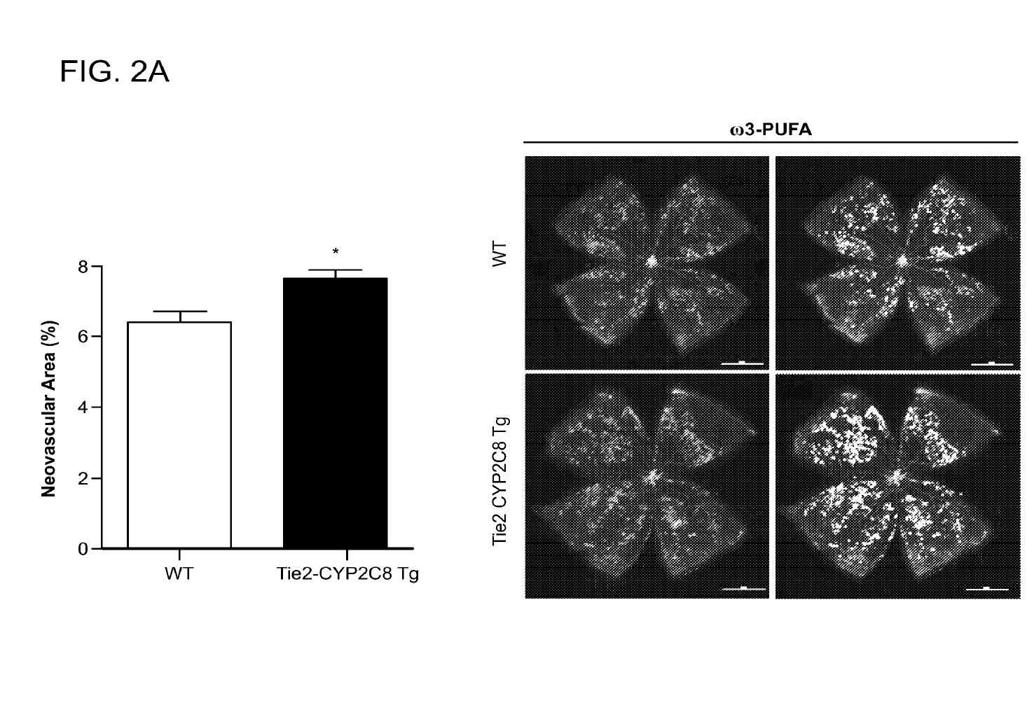

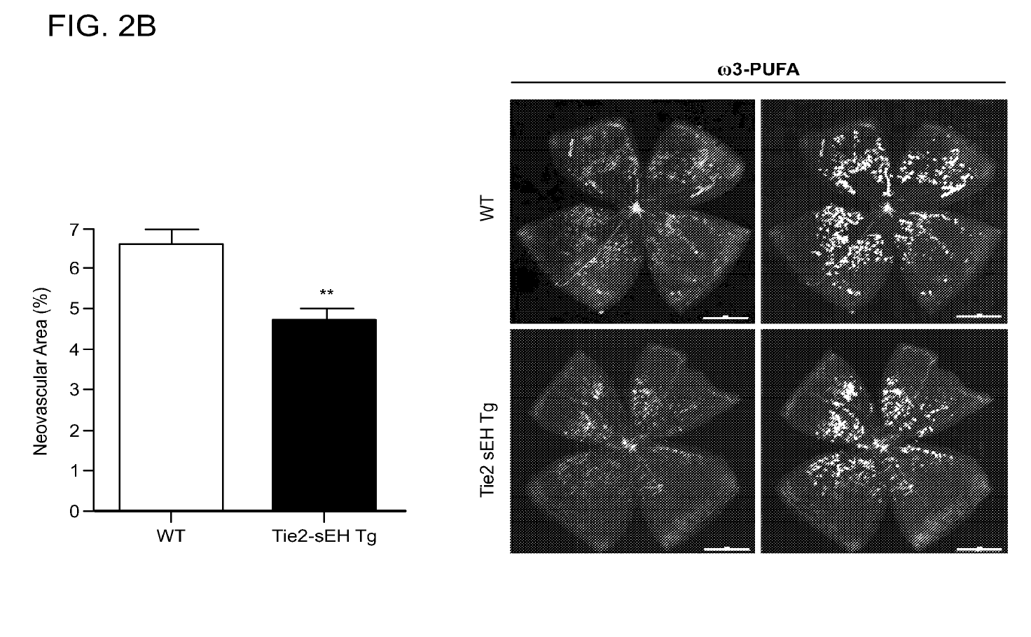

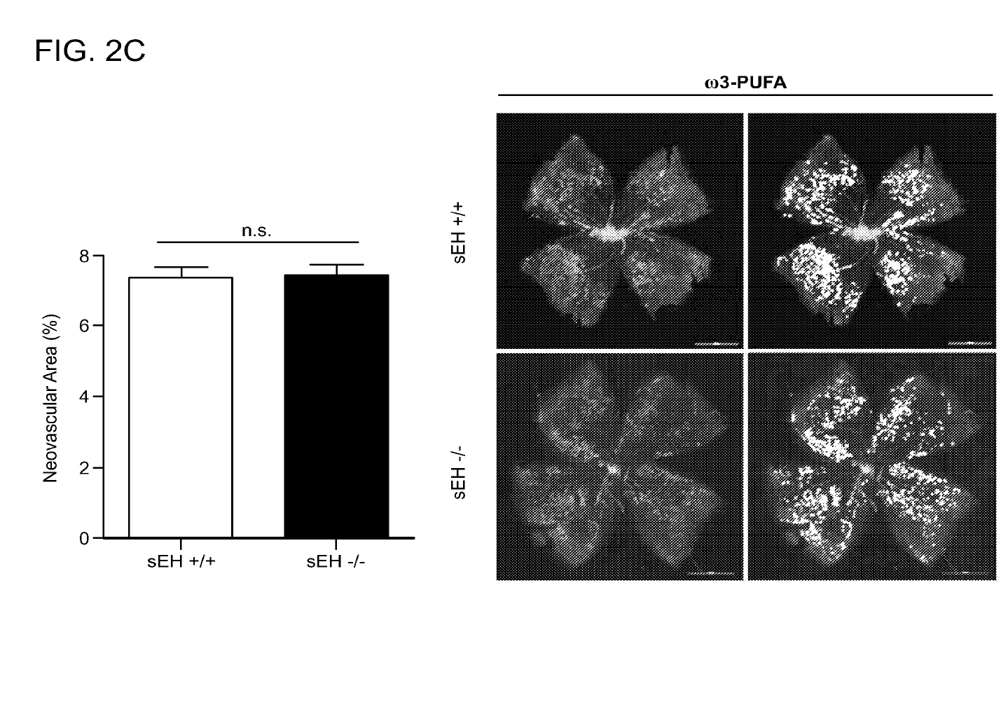

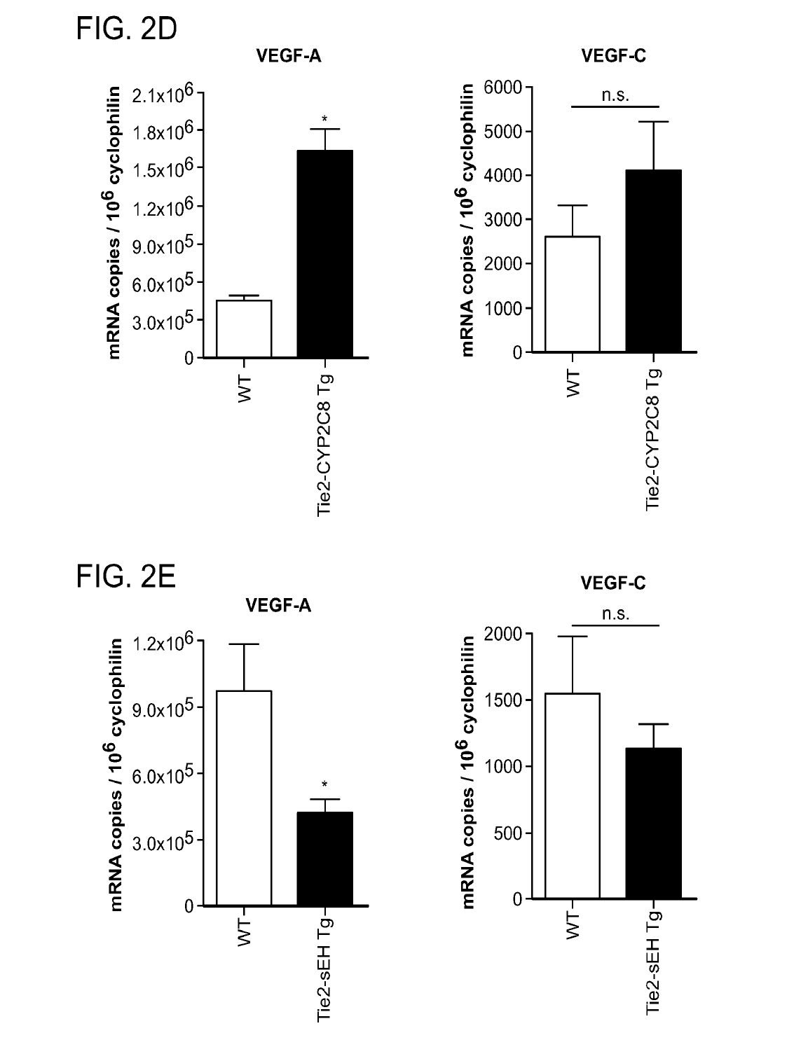

On a ω3PUFA diet, Tie2-CYP2C8-Tg (CYP2C8 overexpressing) mice developed more OIR-neovascularization than WT (7.60±0.29 vs. 6.40±0.33% of total retinal area, p=0.014) (FIG. 2A). Meanwhile, Tie2-sEH-Tg retinas developed less neovascularization versus WT (4.67±0.34 vs. 6.59±0.38%, p=0.0027; FIG. 2B). Germ-line loss of sEH (sEH−/−) had no further effect on neovascularization, as compared to WT (7.39±0.34 vs. 7.35±0.32%, p=0.95; FIG. 2C), likely reflecting an already low sEH expression level in OIR (FIG. 1F).

With ω3PUFA feed, Tie2-CYP2C8-Tg OIR mice had 2.6-fold greater VEGF-A expression than WT (p=0.011), whereas Tie2-sEH-Tg had 57% less VEGF-A expression (p=0.030). No significant difference in VEGF-C level was detected (FIGS. 2D&E).

### Example 3

### In OIR with ω3PUFA Feed, Tie2-CYP2C8-Tg Increased, while Tie2-sEH-Tg Decreased, Plasma Epoxide Levels and Retinal Epoxide:Diol Ratios

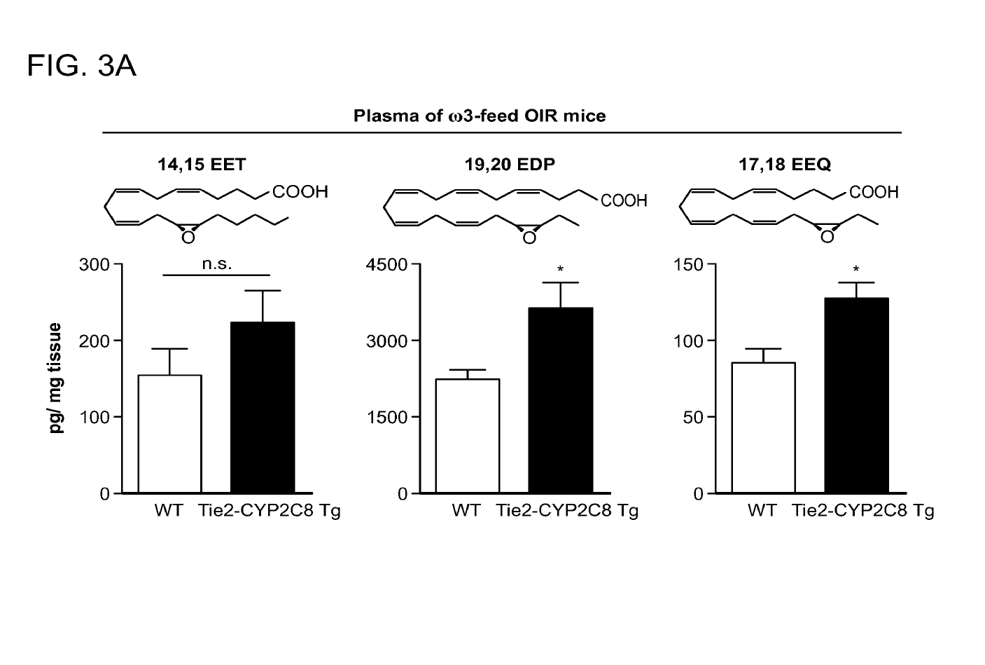

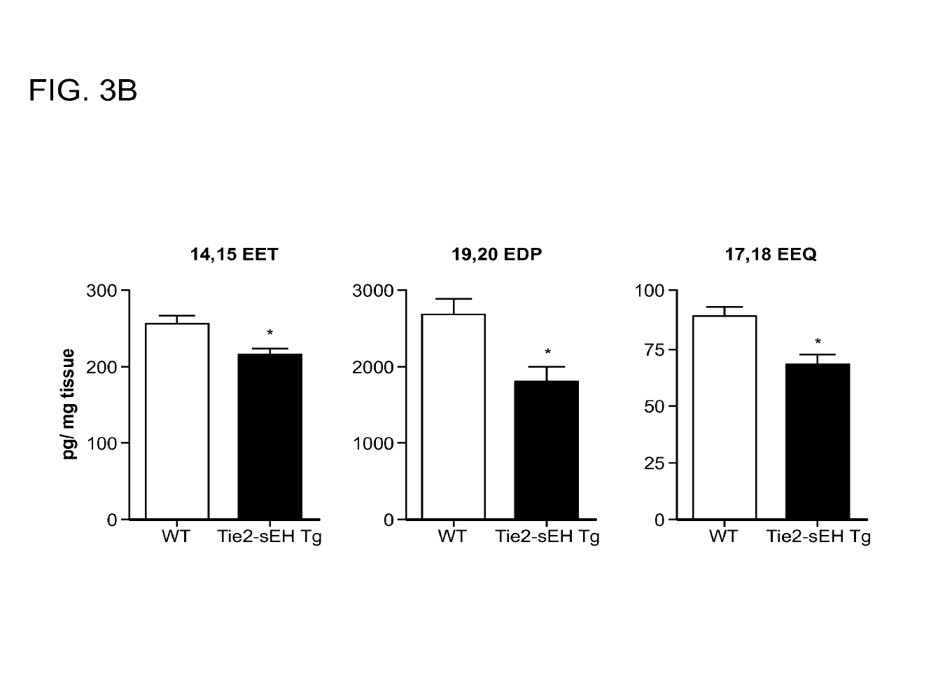

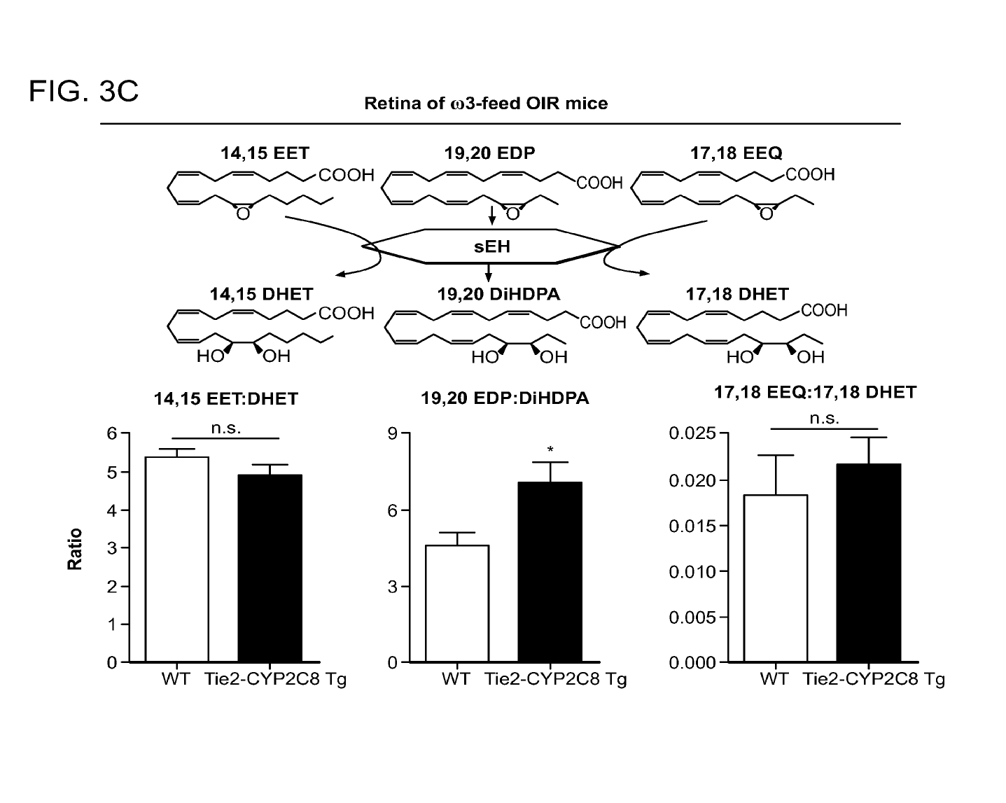

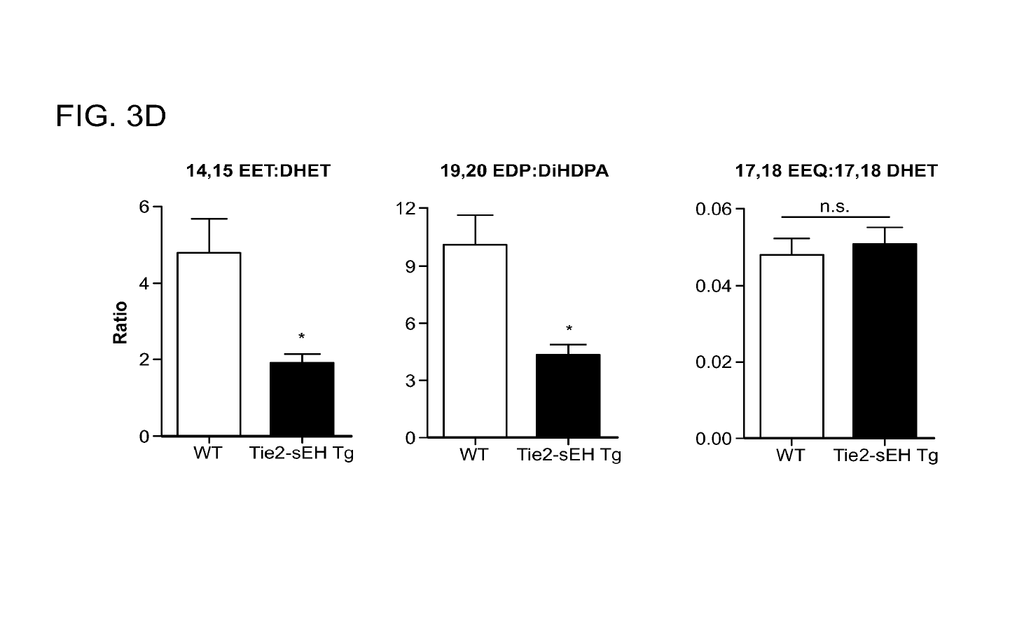

In OIR, plasma from ω3PUFA-fed Tie2-CYP2C8-Tg mice was assessed to have 60% more 19,20-EDP (p=0.029) and 47% more 17,18-EEQ (p=0.030) than WT. The concentration of 19,20-EDP was 30 times higher than 17,18-EEQ in such samples (FIG. 3A). In Tie2-sEH-Tg mice, 19,20-EDP and 17,18-EEQ levels were reduced by 34% (p=0.034) and 24% (p=0.016). The 14,15-EET level was reduced by 16%, p=0.029; FIG. 3B).

In OIR, ω3PUFA-fed Tie2-CYP2C8-Tg retinas have a 52% higher 19,20-EDP:DiHDPA ratio than WT (p=0.045); the 17,18-EEQ:17,18-DHET ratio was unchanged; FIG. 3C). In ω3PUFA-fed Tie2-sEH-Tg retinas, the 19,20-EDP:DiHDPA ratio decreased by 58% (p=0.028); the 17,18-EEQ:17,18-DHET ratio was unchanged. The 14,15-EET:14,15-DHET ratio decreased 60% (p=0.043; FIG. 3D).

### Example 4

### Vascular Sprouting from Tie2-CYP2C8-Tg Aortic Rings Increases with AA or DHA and Tie2-sEH-Tg Sprouting is Suppressed with 19,20-EDP

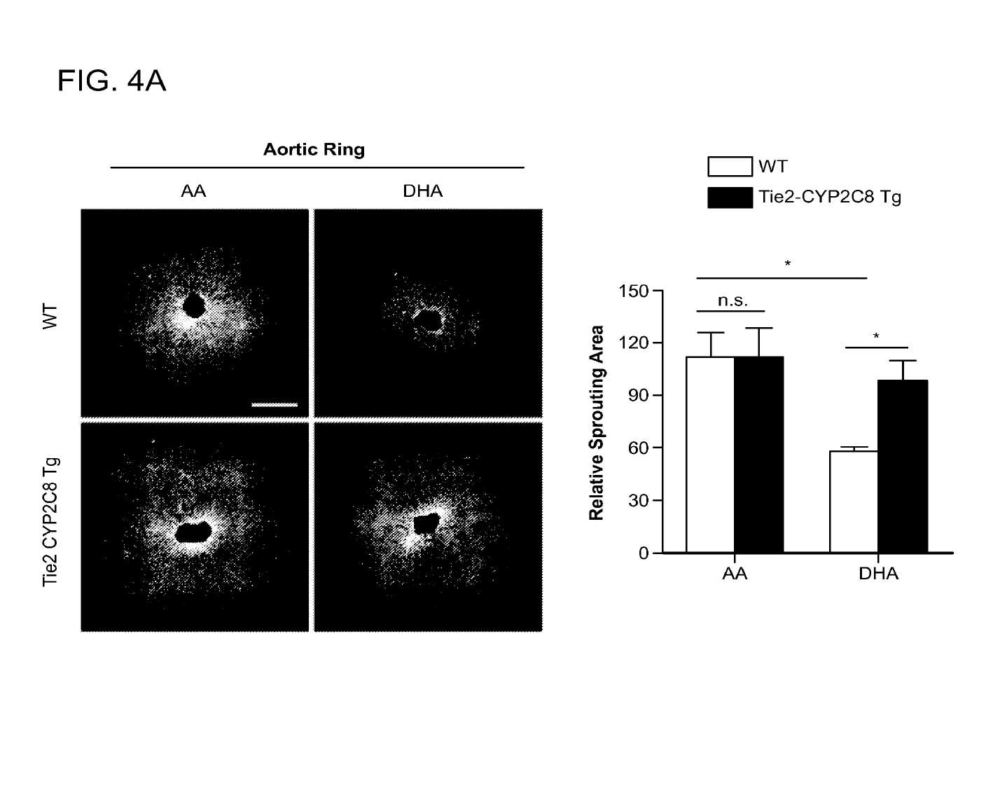

The pro-angiogenic effect of CYP2C8-derived and anti-angiogenic effect of sEH processed ω3PUFA metabolites on angiogenesis were confirmed with an aortic ring-sprouting assay. 30 μM AA (vs. 30 μM DHA) potentiated aortic sprouting in WT (p=0.01), which was abolished in Tie2-CYP2C8-Tg. Tie2-CYP2C8-Tg versus WT had increased sprouting with DHA treatment (p=0.43; FIG. 4A). There was no difference between aortic ring sprouting of WT, Tie2-sEH-Tg and sEH−/− mice treated with 17,18-EEQ, in contrast to 50% less sprouting from 19,20-EDP-treated Tie2-sEH-Tg aortic ring versus WT (p<0.01; FIG. 4B). These results confirmed that Tie2-CYP2C8-Tg promoted angiogenesis with ω3PUFA and indicated that decreased neovascularization in Tie2-sEH-Tg was directly attributable to accelerated degradation of 19,20-EDP by over-expressed sEH.

### Example 5

### In OIR, ω6PUFA Feed Increased Neovascularization in Tie2-CYP2C8-Tg Mice

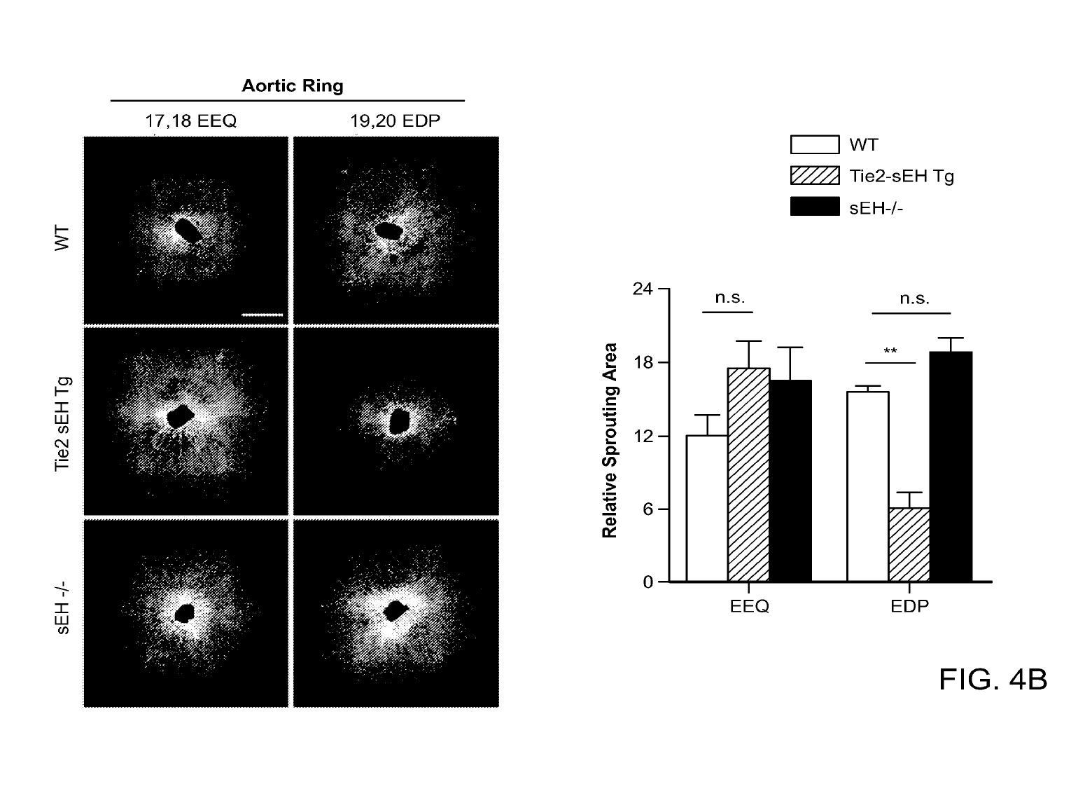

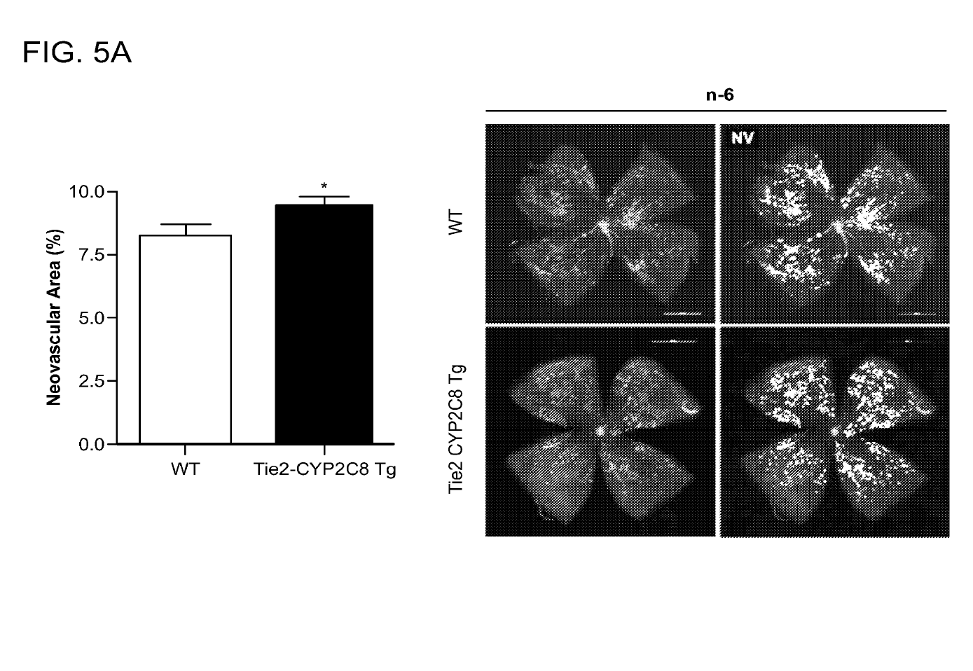

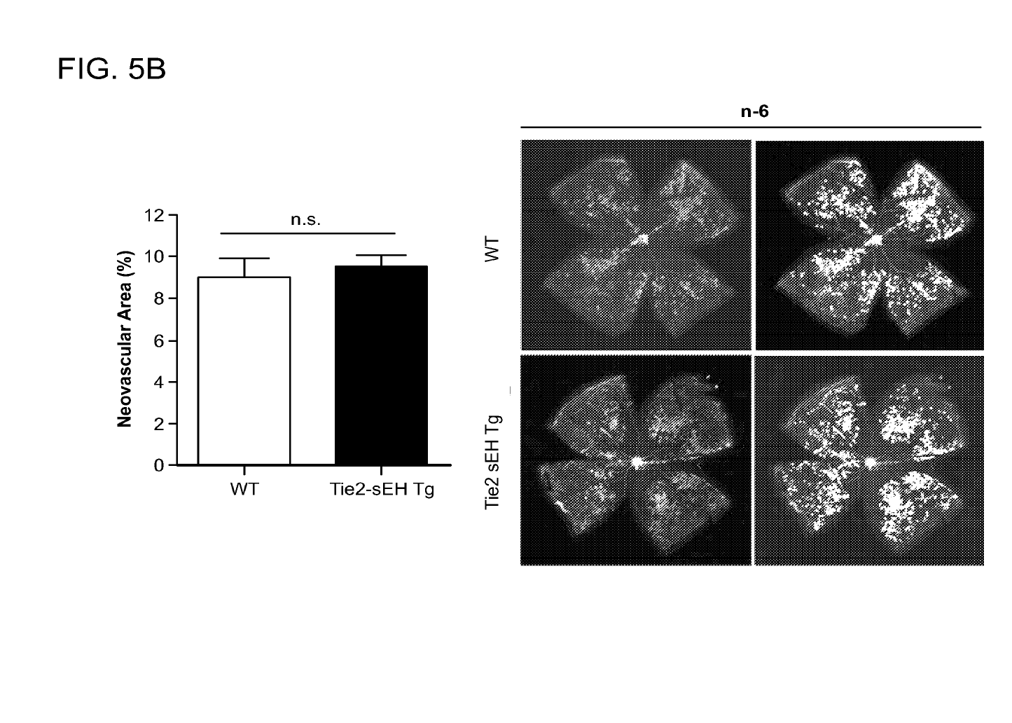

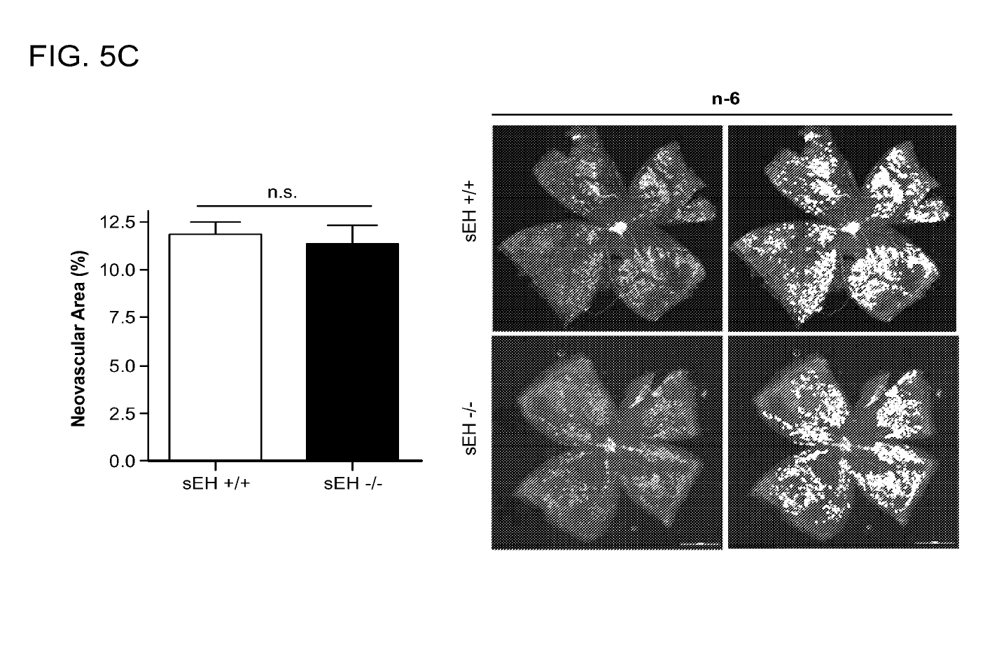

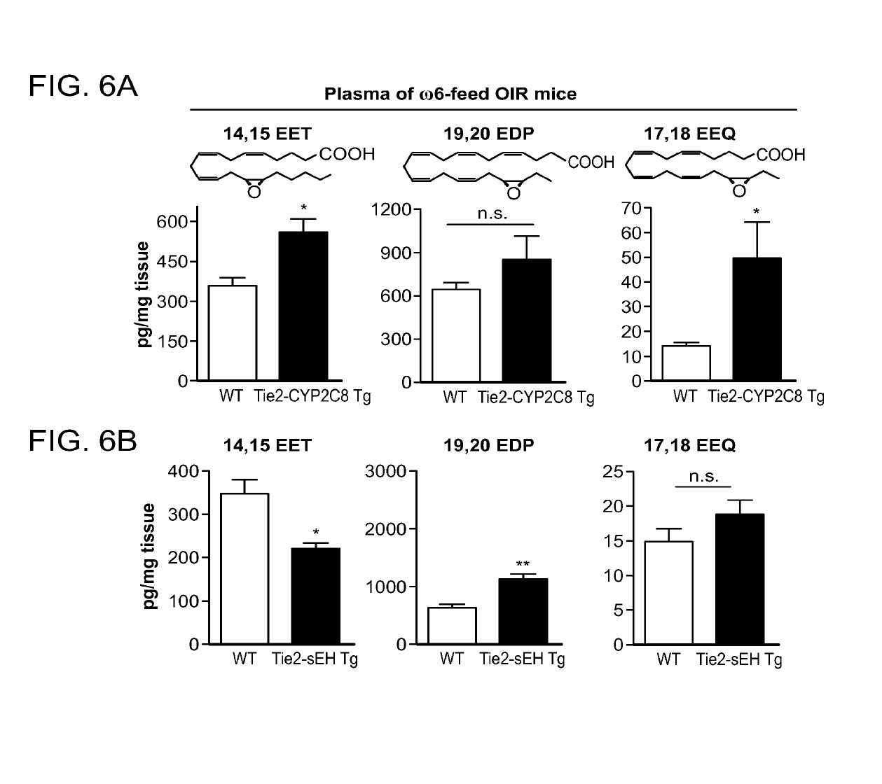

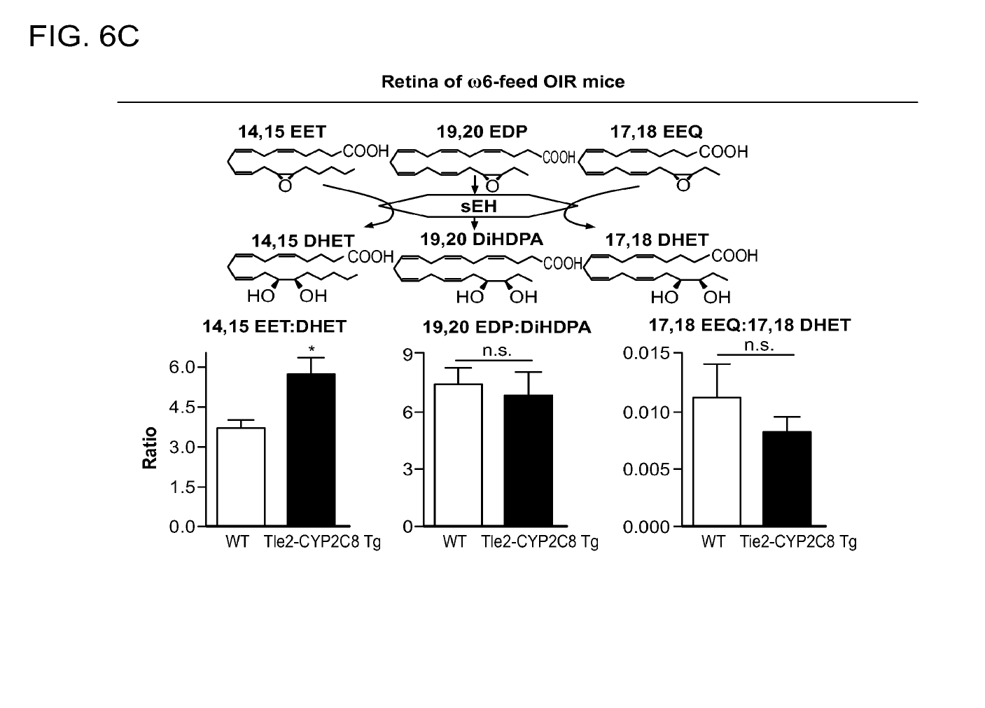

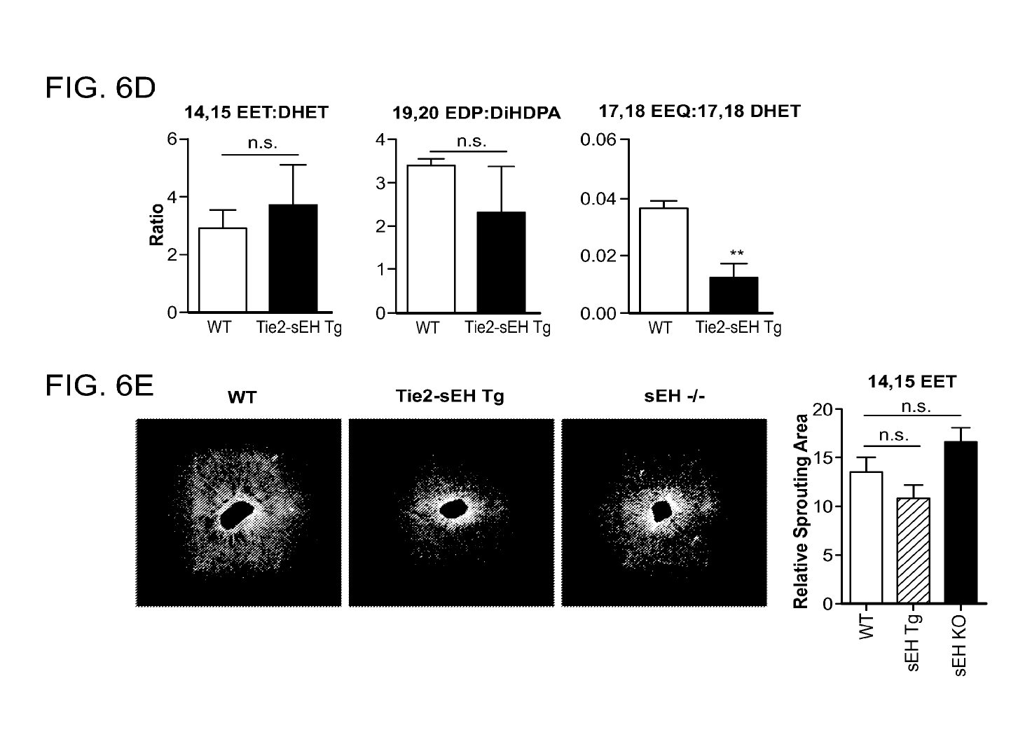

With ω6PUFA-feed, Tie2-CYP2C8-Tg induced OIR-neovascularization versus WT (9.458±0.3425 vs. 8.291±0.3979, p=0.032). In contrast, no difference was seen with Tie2-sEH-Tg or sEH−/− (FIG. 5). Plasma 14,15-EET levels and the retinal 14,15-EET:14,15-DHET ratio increased with Tie2-CYP2C8-Tg versus WT, consistent with increased neovascularization (FIG. 6A-D). With 14,15-EET treatment, aortic ring sprouting was similar to that observed in the Tie2-sEH-Tg, sEH−/− and WT mice (FIG. 6E).

### Example 6

### Identification of Fenofibrate as a Therapeutically Effective CYP2C8 Inhibitor

Fenofibrate has been previously described as a cholesterol lowering drug that reduces lipid levels in a subject via activation of peroxisome proliferator-activated receptor alpha (PPARα). Specifically, PPARα has been described to activate lipoprotein lipase and reduce apoprotein CIII, resulting in increased lipolysis and elimination of triglyceride-rich particles from plasma (Staels et al. Circulation 98: 2088-93). To examine the efficacy and mechanism of fenofibrate as a therapeutic for vascular diseases of the retina, fenofibrate was administered by gavage (GV) to mice as detailed below, resulting in the identification of fenofibrate as a suppressor of neovascularization (NV) in oxygen-induced retinopathy (which was increased in Cyp2C8 Tg mice), via inhibition of Cyp2C8 activity.

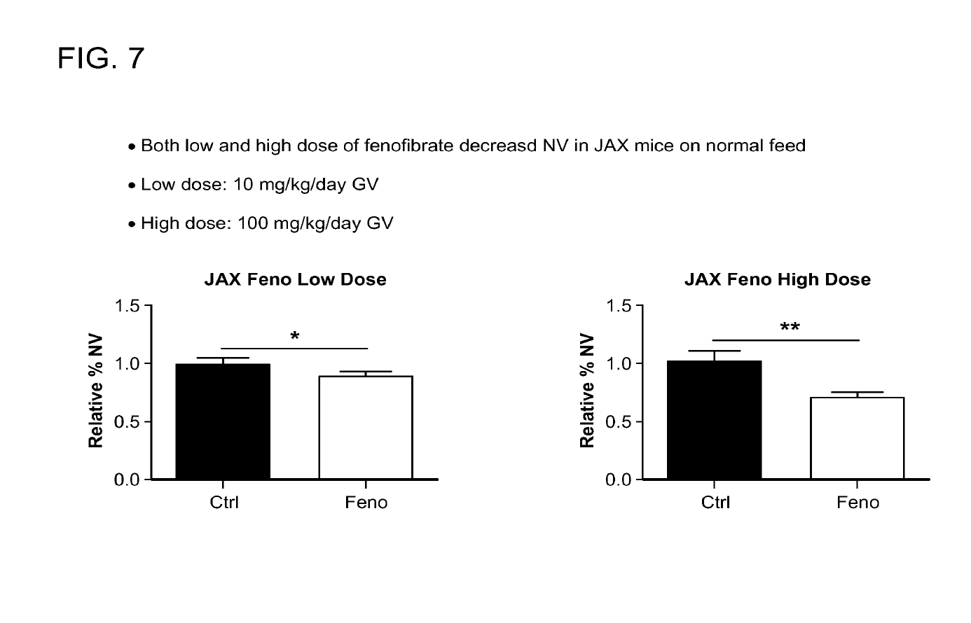

As shown in FIG. 7, when fenofibrate was administered by gavage (GV) to JAX mice (WT) receiving normal feed, neovascularization was observed to be reduced in a statistically significant manner. Notably, both low (10 mg/kg/day GV) and high (100 mg/kg/day GV) levels of fenofibrate were observed to produce a significant reduction in neovascularization (NV) in such mice. Because PPARα-related effects of were expected to be induced only at high doses of fenofibrate, the effect of low dose fenofibrate on neovascularization was surprising, and implicated a PPARα-independent mode of action for fenofibrate in inhibition of neovascularization.

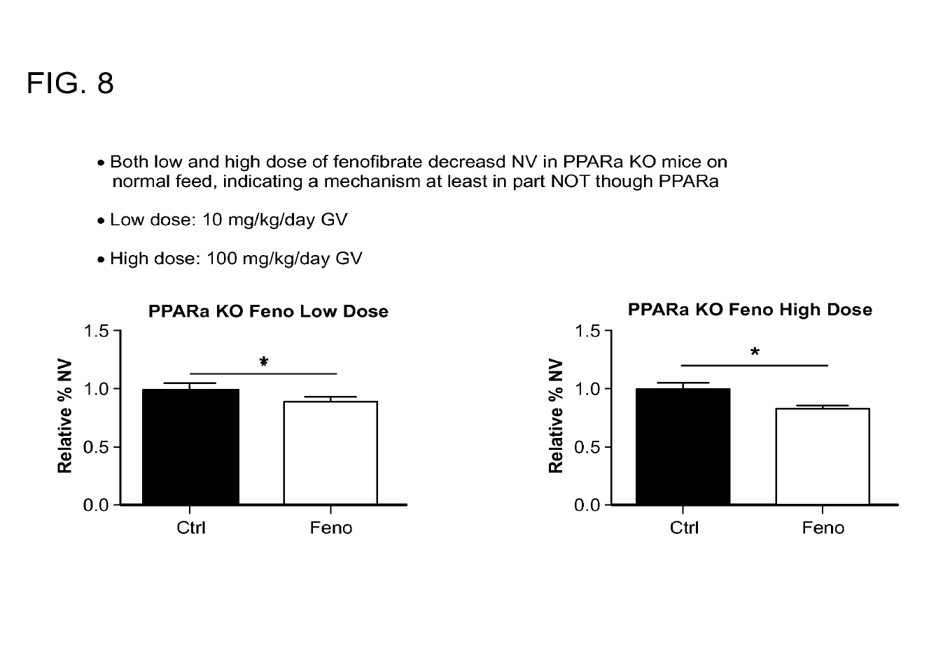

To verify whether at least some effects of fenofibrate were indeed PPARα-independent, inhibition of neovascularization was assessed in PPARα knockout mice administered fenofibrate. As shown in FIG. 8, results similar to those observed in JAX (WT) mice were obtained in PPARα knockout mice. Specifically, both low (10 mg/kg/day GV) and high (100 mg/kg/day GV) levels of fenofibrate were observed to produce a significant reduction in neovascularization (NV) in PPARα knockout mice on normal feed. Thus, the observed effects of fenofibrate in inhibiting NV were confirmed to be PPARα-independent.

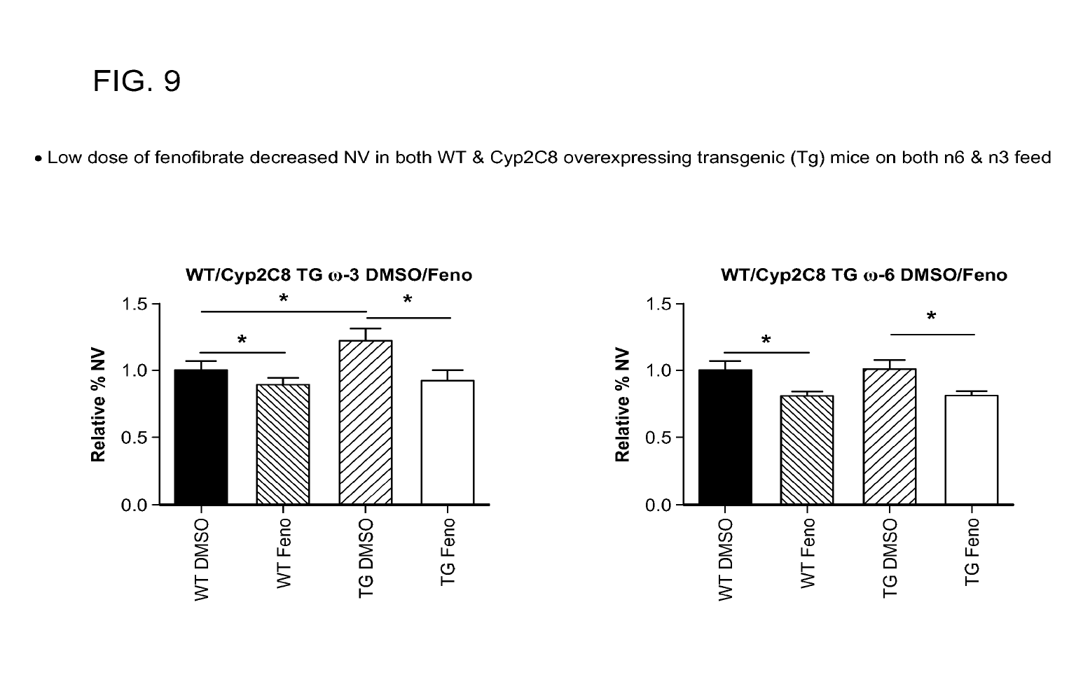

To examine whether fenofibrate was acting via modulation of CYP2C8 to reduce NV, the impact of administering fenofibrate to mice overexpressing CYP2C8 (Cyp2C8 transgenic mice, “Cyp2C8 Tg”) was examined. As shown in FIG. 9, the magnitude of the extent of reduction in NV observed in mice administered low dose fenofibrate (10 mg/kg/day GV) was enhanced in mice that overexpressed CYP2C8, as compared to the magnitude of reduction observed in corresponding WT mice. Similar results (inhibition of NV being enhanced in CYP2C8 overexpressing mice) were observed for mice fed ω3 (n3) or ω6 (n6), indicating that both ω3 and ω6 pathways were involved in these CYP2C8-dependent results.

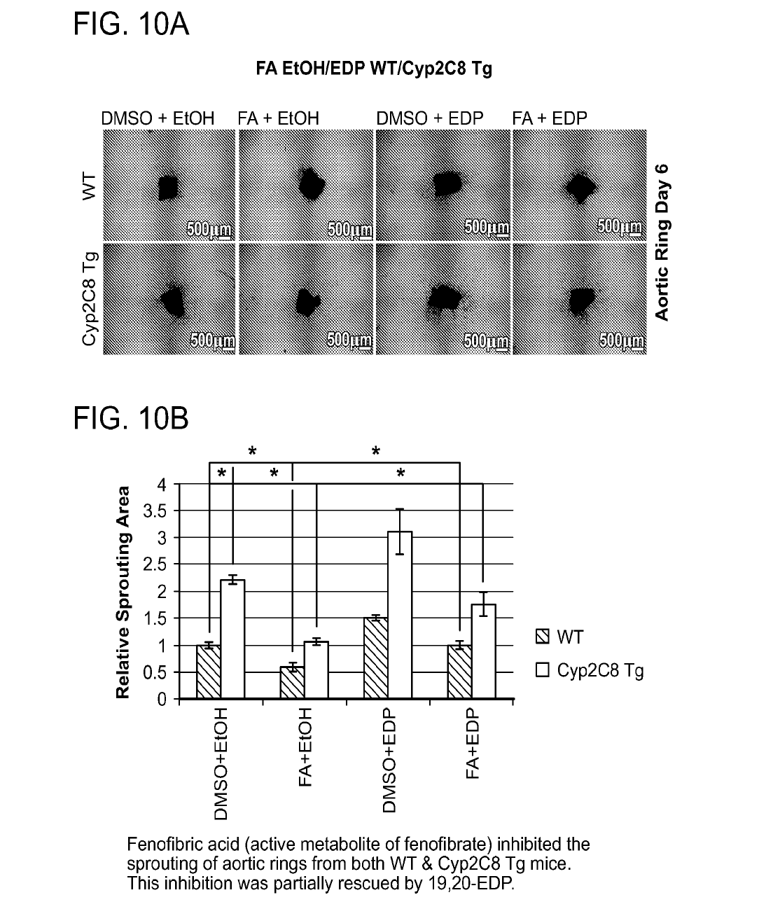

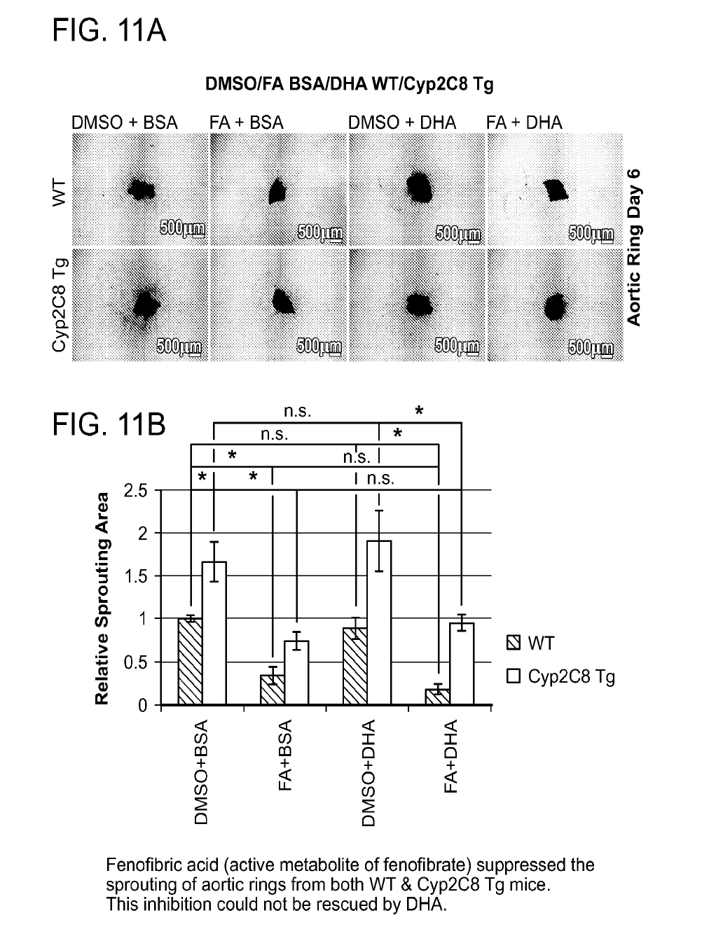

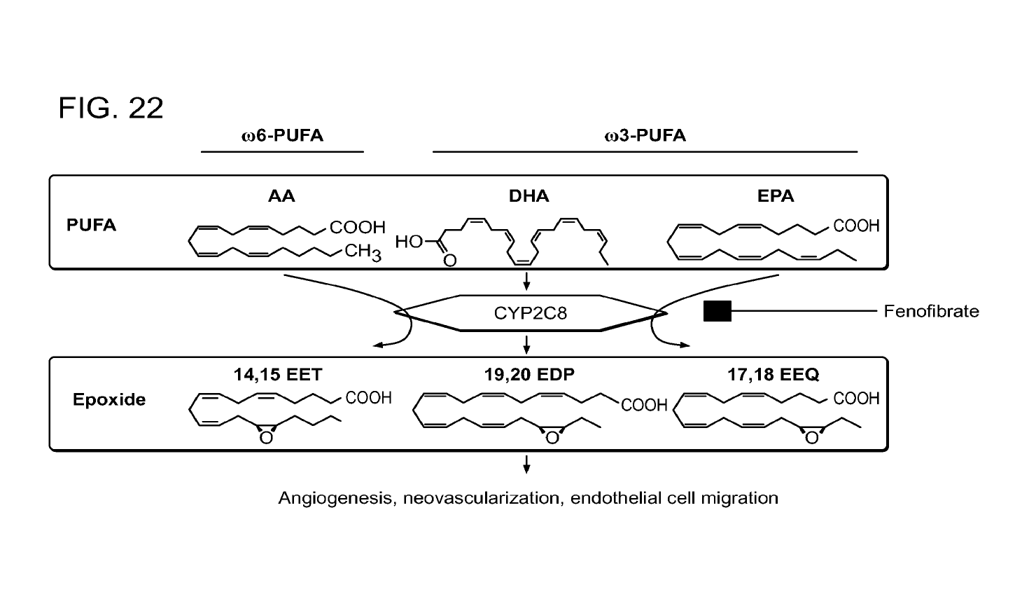

The mechanism of the effect observed for fenofibrate was examined further in aortic ring sprouting assays. As shown in FIG. 10, fenofibric acid (FA, the active metabolite of fenofibrate) was observed to inhibit the sprouting of aortic rings from both WT & Cyp2C8 Tg mice. Consistent with this result being attributable to inhibition of Cyp2C8 by FA, this inhibition was partially rescued by 19,20-EDP (a post-CYP2C8 metabolite of DHA, as shown in FIG. 22 below). Indeed, as shown in FIG. 11, no rescue of this FA inhibition of aortic ring sprouting was observed when DHA, rather than 19,20-EDP was administered, indicting the involvement of post-CYP2C8 metabolites—and inhibition of the Cyp2C8 enzyme—in the NV/aortic ring growth process that was blocked by FA acting as a CYP2C8 inhibitor.

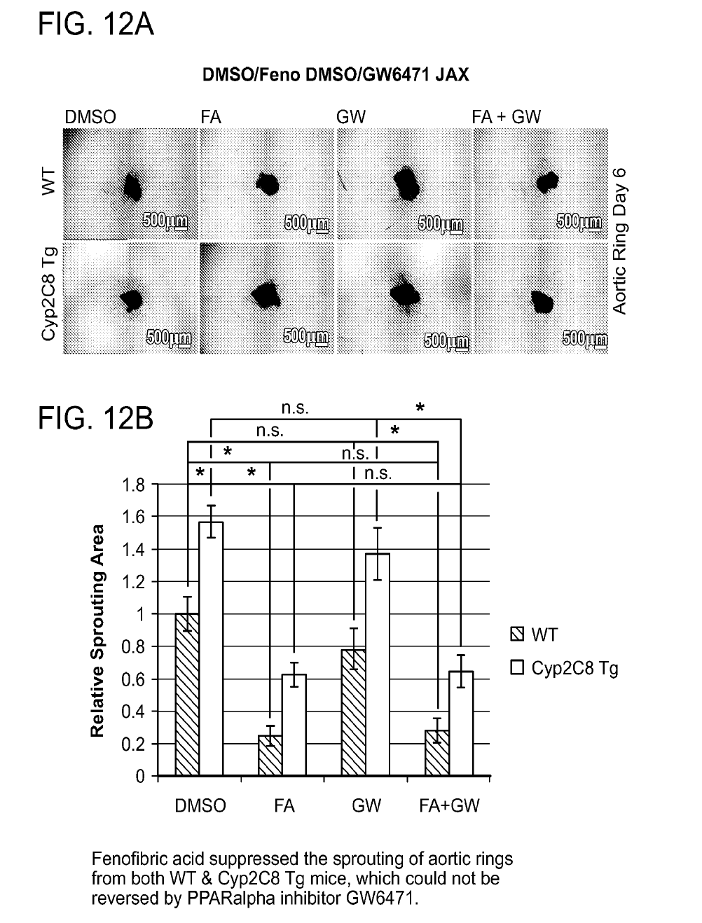

In addition, as shown in FIG. 12, the effect of FA acting to inhibit aortic ring sprouting in both WT and Cyp2C8 Tg mice was confirmed as PPARα-independent, when PPARα inhibitor GW6471 was examined and found to have no impact upon the observed effect of FA on aortic ring sprouting.

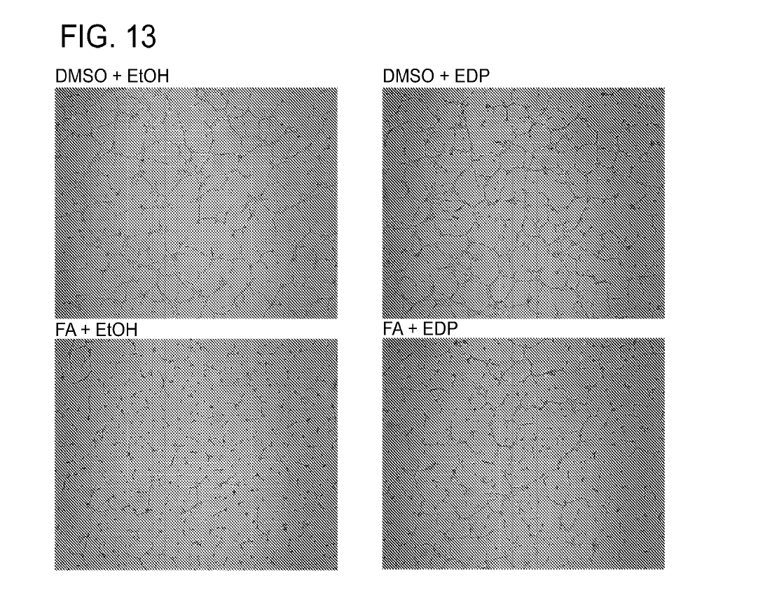

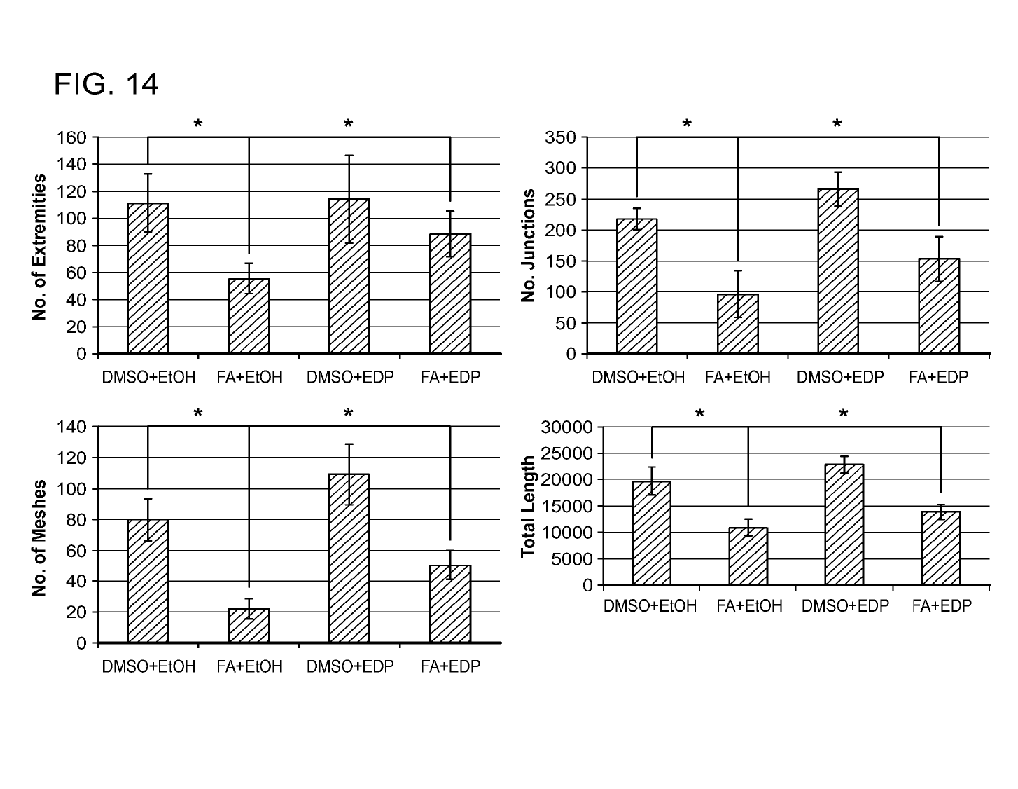

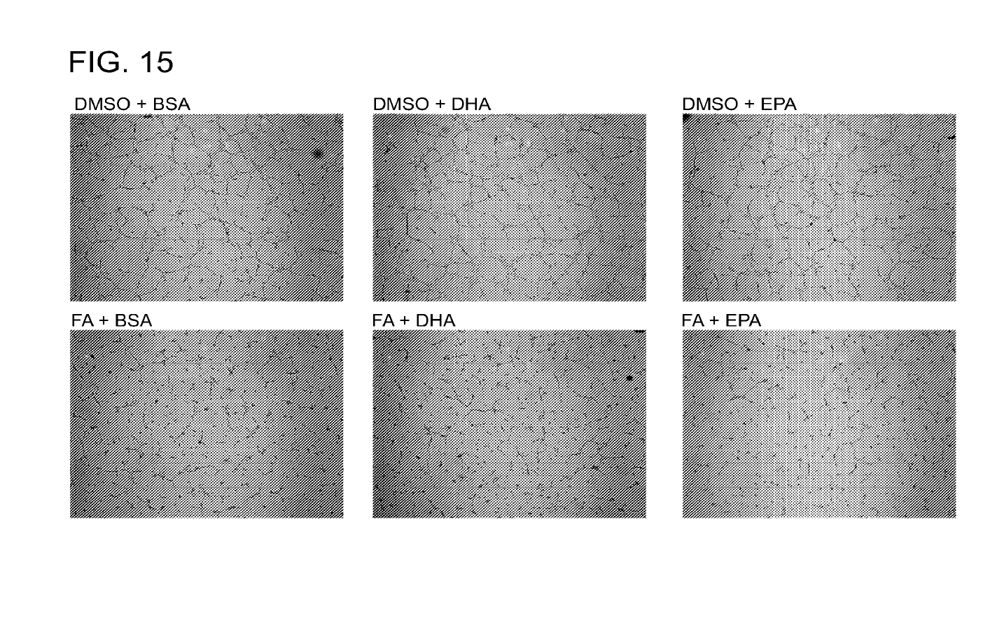

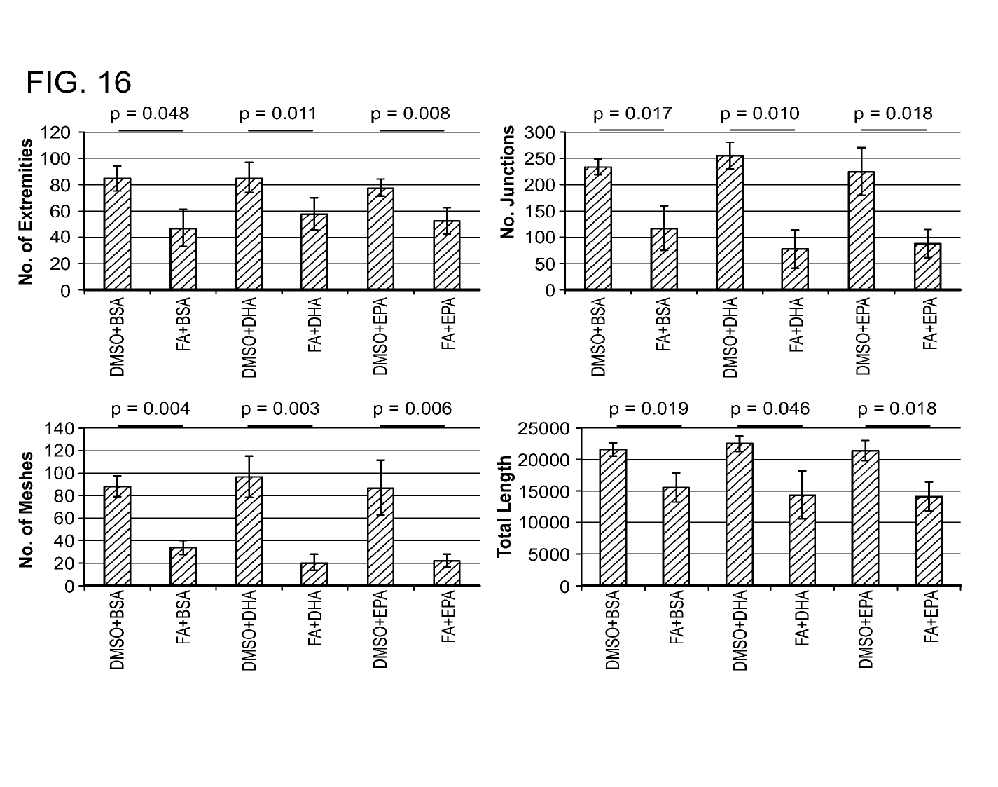

Having confirmed the effect of fenofibrate in reducing neovascularization (NV) and in reducing aortic ring sprouting, human retinal microvascular endothelial cells (HRMEC) were then examined for a corresponding series of effects on tubule formation. As shown in FIG. 13, FA was observed to inhibit HRMEC tubule formation, and this effect was partially rescued by 19,20 EDP, as described for FA and the aortic ring assay in FIG. 10 above. These results were quantitated and are presented as histograms in FIG. 14, where 19,20 EDP (omega 3 metabolite of CYP2C8) partially rescued the inhibition of HRMEC tubule formation by FA. As shown in FIG. 15, akin to DHA having been identified as unable to rescue the aortic ring sprouting effect observed for fenofibrate, another compound upstream of CYP2C8, w3LCPUFA, was found to be unable to rescue the inhibition of HRMEC tubule formation by FA. In FIG. 16, the results of such experiments were quantitated and are presented in histogram format.

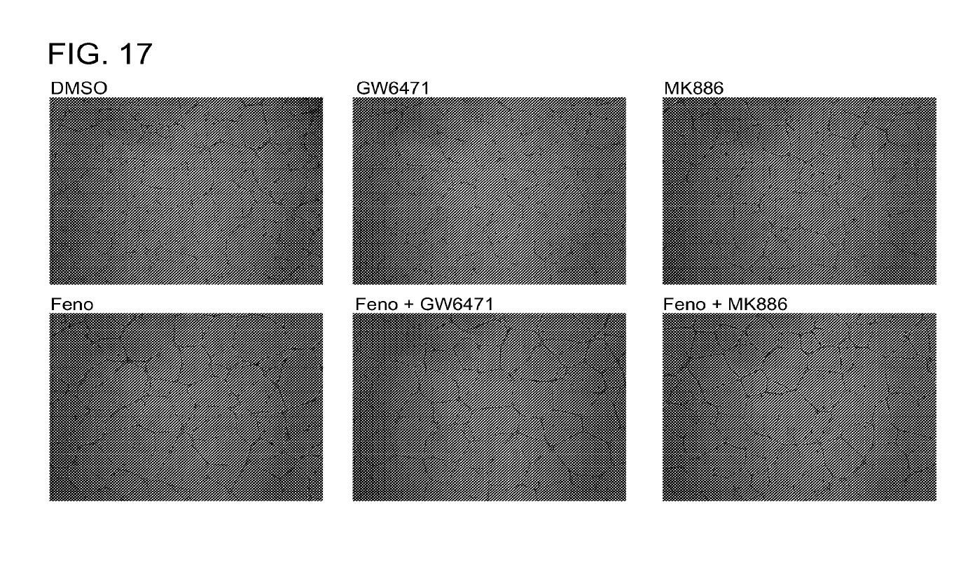

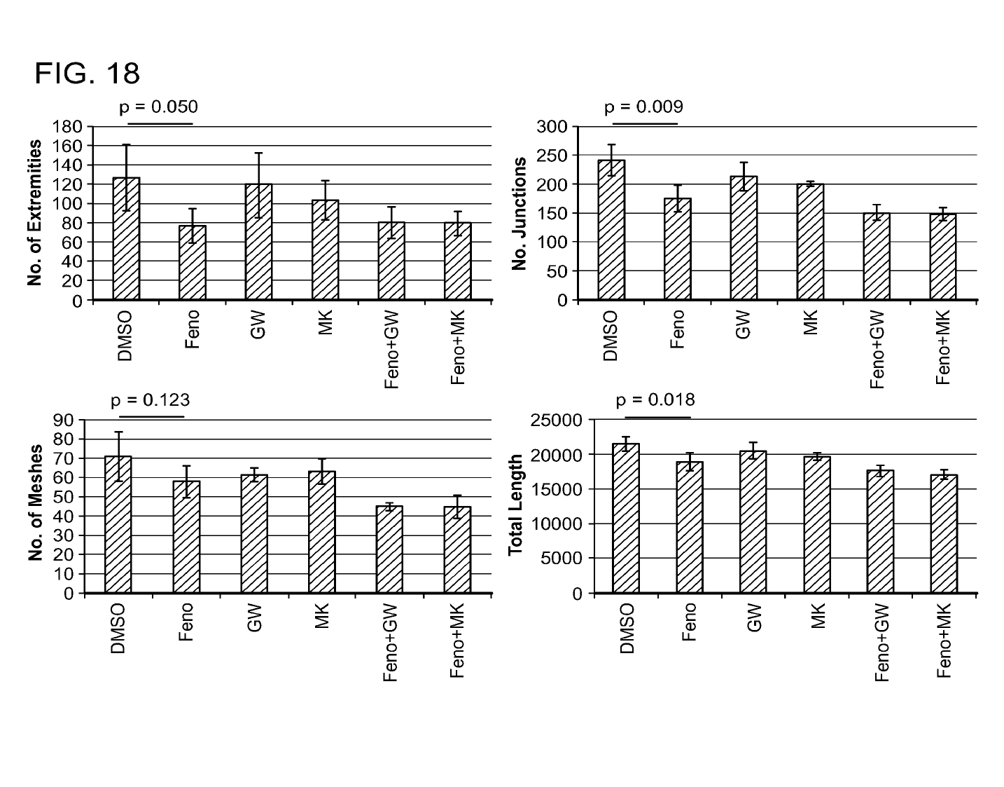

As shown in FIGS. 17 and 18, fenofibrate was identified to inhibit HRMEC tubule formation in a manner that was PPARα-independent, when PPARα inhibitor GW6471 was examined and found to have no impact upon the observed effect of fenofibrate on HRMEC tubule formation.

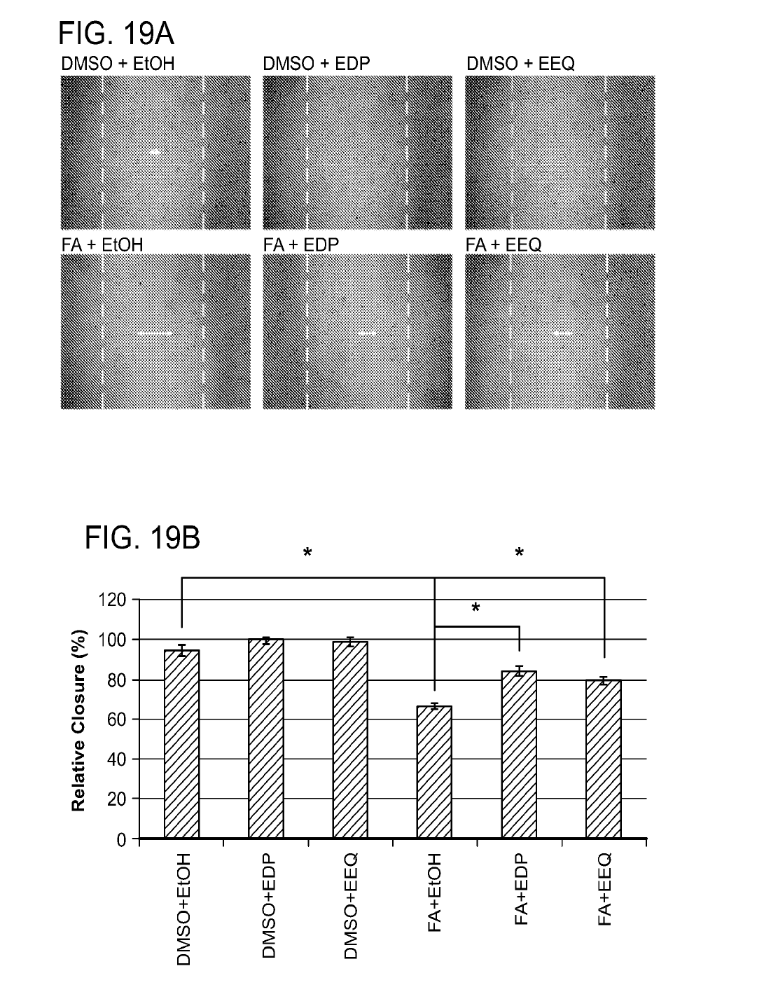

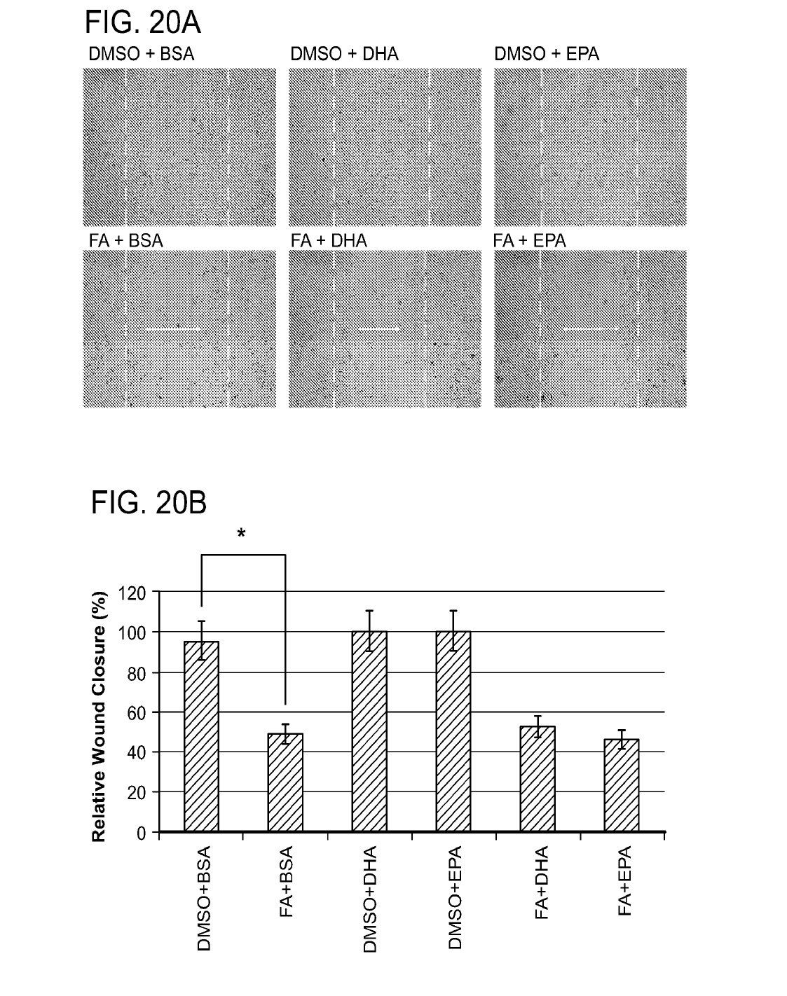

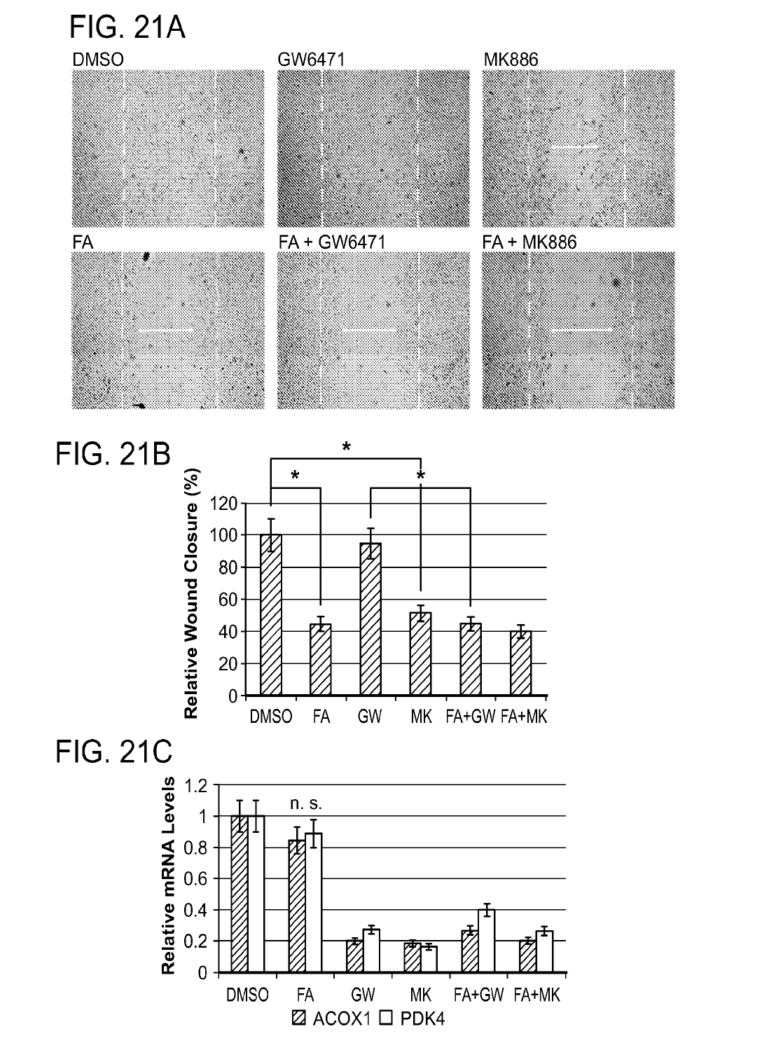

In contrast to compounds upstream of the CYP2C8 enzyme (DHA, EPA, w3LCPUFA), compounds downstream of the CYP2C8 enzyme continued to be identified as having at least partial rescue qualities. In FIG. 19, 19,20 EDP and 17,18 EEQ (a compound downstream of EPA and CYP2C8—see FIG. 22) were identified as partially rescuing the inhibition of HRMEC migration by FA. In FIG. 20, w3LCPUFA was identified as incapable of rescuing HRMEC migration by FA. In FIG. 21, the FA inhibition of HRMEC migration was observed to be PPARα-independent, when PPARα inhibitor GW6471 was examined and found to have no impact upon the observed effect of fenofibrate on HRMEC migration. FIG. 22 shows the assessed site of action of fenofibrate/FA within the ω3 and w6 pathways.

### Example 7

### Identification of Montelukast as a Therapeutically Effective CYP2C8 Inhibitor

Montelukast is a leukotriene receptor antagonist (LTRA) that has previously been used for the maintenance treatment of asthma, and to relieve symptoms of seasonal allergies in a subject (Lipkowitz et al. The Encyclopedia of Allergies (2nd ed.)). Montelukast comes as a tablet, a chewable tablet, and granules to take by mouth, and is usually taken once a day with or without food. Montelukast is primarily recognized as a CysLT1 antagonist; in blocking the action of leukotriene D4 (and secondary ligands LTC4 and LTE4) on the cysteinyl leukotriene receptor CysLT1 in the lungs and bronchial tubes by binding to it. Without wishing to be bound by theory, this is thought to reduce the the bronchoconstriction otherwise caused by the leukotriene, resulting in less inflammation.

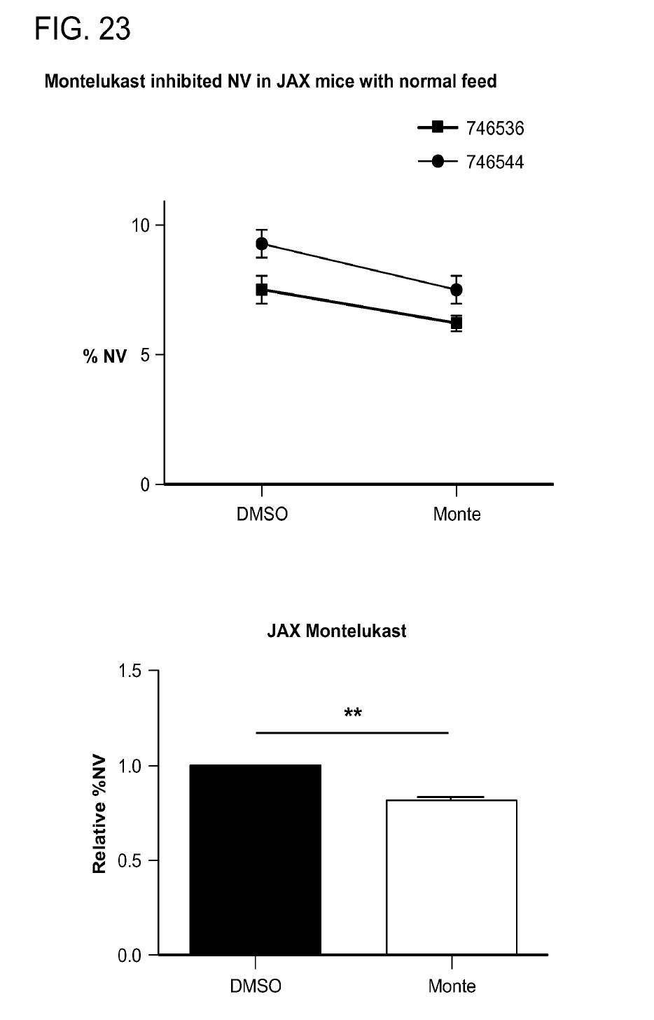

In the current example, montelukast was newly identified to behave as an inhibitor of CYP2C8, with effects paralleling those observed for fenofibrate above. Specifically, as shown in FIG. 23, when montelukast was administered to JAX mice (WT) receiving normal feed, neovascularization was observed to be reduced in a statistically significant manner.

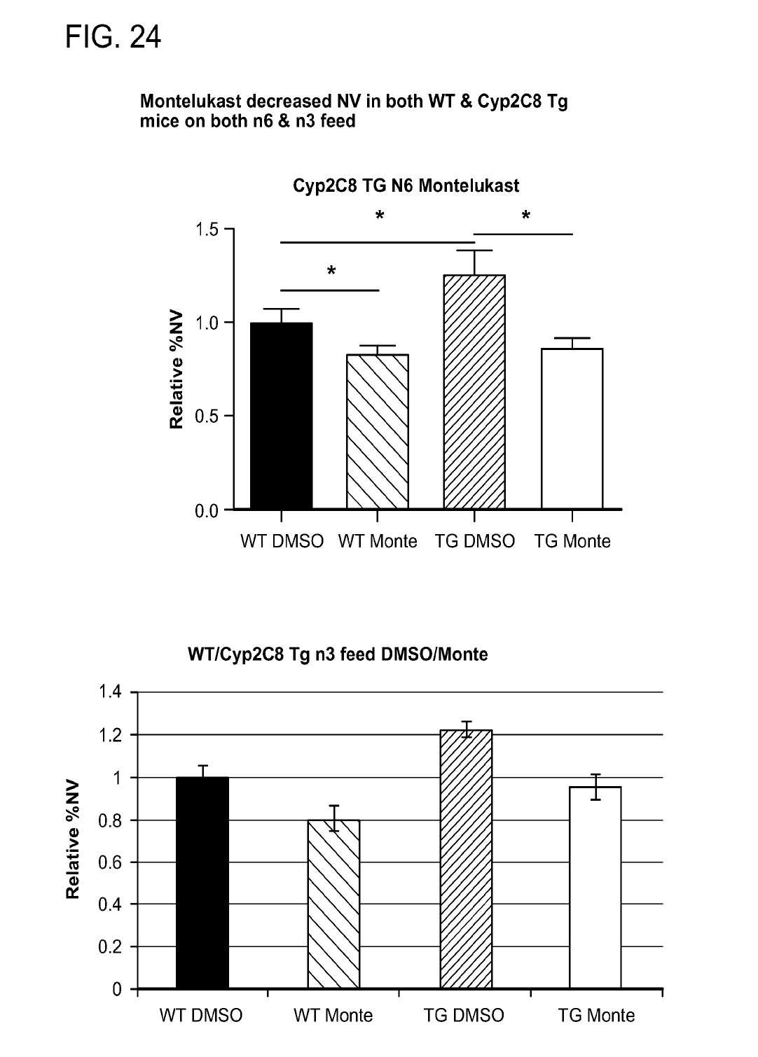

As shown in FIG. 24, the action of montelukast occurring via modulation of CYP2C8 to reduce NV was confirmed by examining the impact of administering montelukast to mice overexpressing CYP2C8 (Cyp2C8 transgenic mice, “Cyp2C8 Tg”). As for fenofibrate above, the magnitude of the extent of reduction in neovascularization (NV) observed in mice administered montelukast (10 mg/kg/day GV) was enhanced in mice that overexpressed CYP2C8, as compared to the magnitude of reduction observed in corresponding WT mice. Similar results (inhibition of NV being enhanced in CYP2C8 overexpressing mice) were observed for mice fed ω3 (n3) or ω6 (n6), indicating that both ω3 and ω6 pathways were involved in these CYP2C8-dependent results for montelukast.

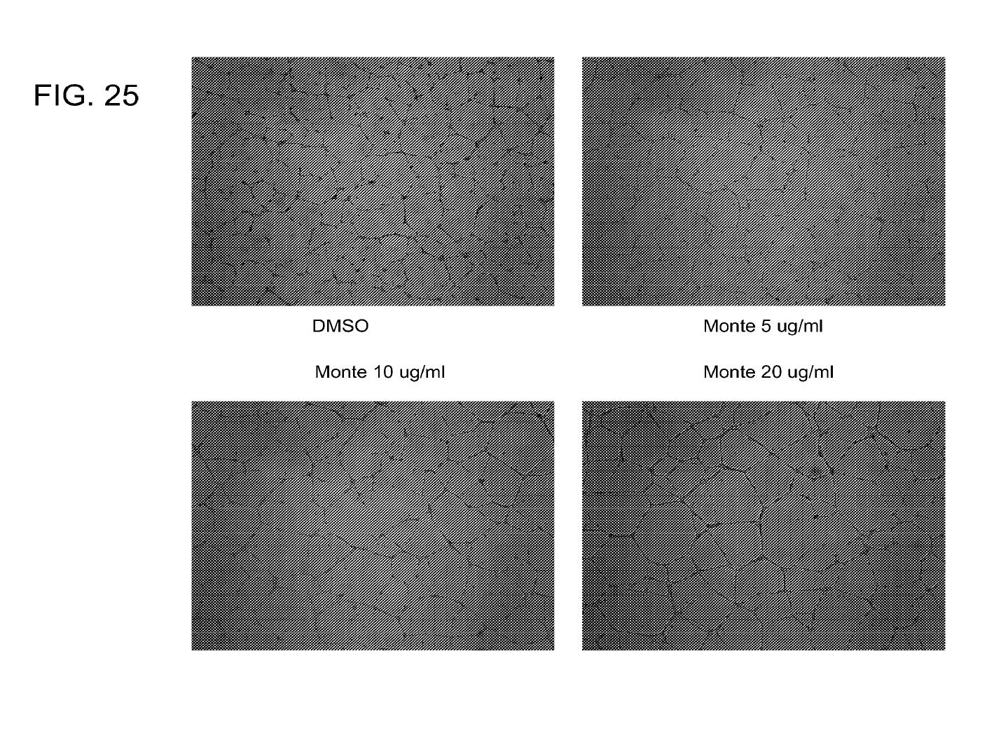

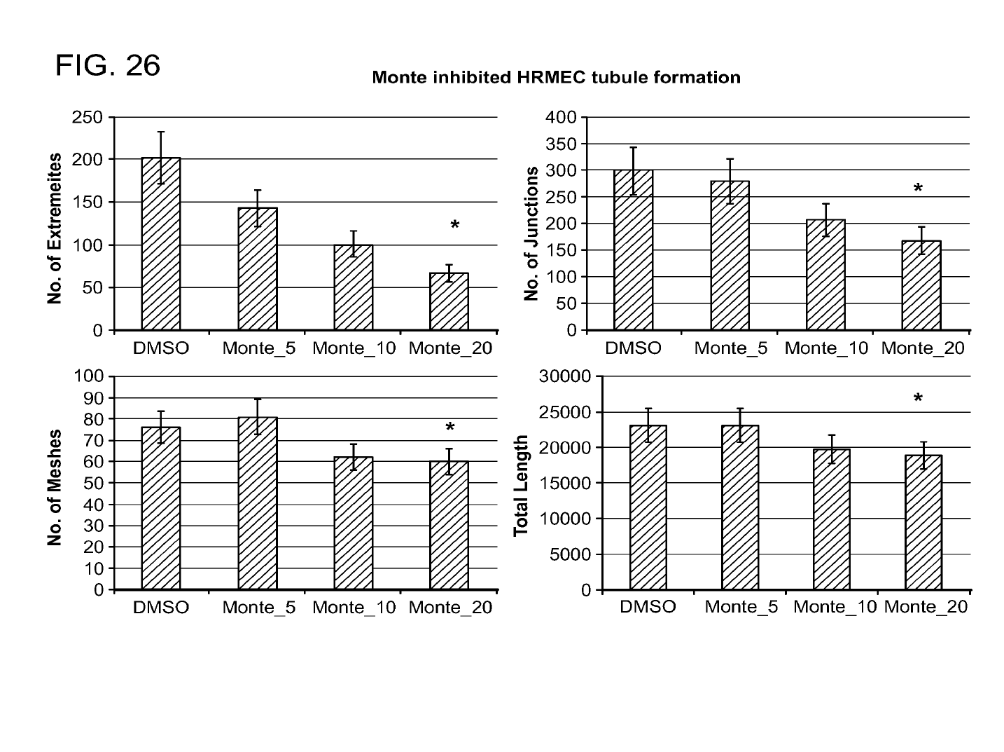

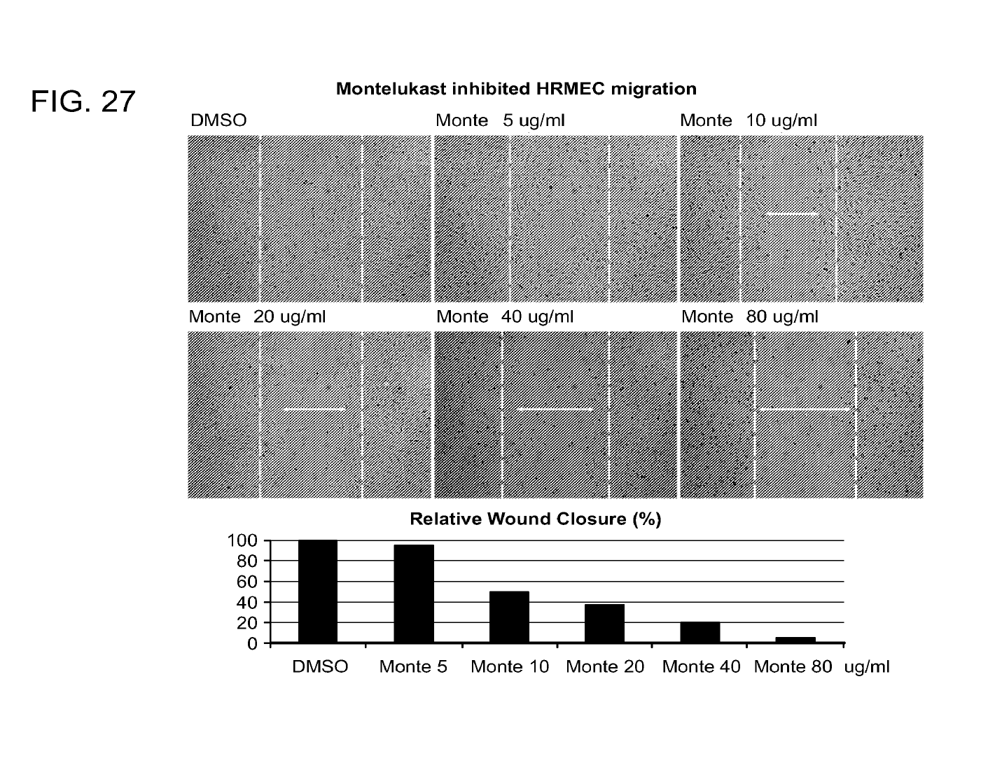

FIGS. 25 and 26 demonstrate that the effects of montelukast on HRMEC tubule formation showed clear dose-response curves, with results also paralleling those observed for fenofibrate, while in FIG. 27, HRMEC migration was observed to be inhibited by montelukast, in a manner that also showed a clear dose-response curve. Thus, montelukast exhibited effects similar to fenofibrate in all assays examined, indicating that both montelukast and fenofibrate were therapeutically effective inhibitors of CYP2C8.

In additional montelukast experiments, aortic ring assays are also performed, akin to those performed above for fenofibrate.

Finding new approaches to treat retinopathy is important. It has been established that w3PUFA feed, overall, in OIR, reduces neovascularization via COX and LOX anti-angiogenic metabolites. Described herein is a novel role of CYP2C8 and sEH in ω3PUFA-mediated retinopathy in that CYP2C8 overexpression (Tie2-driven) potentiates neovascularization with ω3PUFA feed primarily by increasing plasma DHA-derived 19,20-EDP and the retinal 19,20-EDP:DiHDPA ratio. EPA-derived EEQ concentrations are 30-fold lower. Tie2-driven sEH overexpression with ω3PUFA feed decreases neovascularization not only through reduction in plasma 19,20-EDP and the retinal 19,20-EDP:DiHDPA ratio, but also reduction in plasma levels of the pro-angiogenic AA-derived 14,15-EET and the retinal 14,15-EET:14,15-DHET ratio. In wild-type mice CYP2C is induced (primarily in macrophages and leukocytes) and sEH is reduced in OIR, increasing the level of 19,20-EDP.

A recent study found that EDPs inhibit EC migration and tumor angiogenesis by suppressing VEGF-C with no impact on VEGF-A¹². In retina, increased VEGF-A was found, but no change was found in VEGF-C expression in ω3PUFA-fed Tie2-CYP2C8-Tg and decreased VEGF-A expression in Tie2-sEH-Tg was found, consistent with their observed neovascular phenotypes in OIR. These results suggest complex crosstalk among AA, DHA and EPA metabolites and metabolizing enzymes. Overexpression of CYP2C may induce COX-2¹⁴ and stabilization of 14,15-EET may reduce the expression of 5-LOX¹⁵, all impacting active PUFA metabolite levels. In addition, 19,20-EDP may have a different angiogenic function depending on tissue-specific expression of CYP2C8 and sEH. Cardiomyocytes expressing CYP2C8 increase recovery after cardiac ischemia/reperfusion. However, ECs expressing CYP2C8 reduce recovery⁷. In OIR retina, leukocyte-derived EETs can induce leukocyte-EC adhesion¹⁶, and may cause infiltration of Cyp2C-positivemonocytes/macrophages. Further studies on the interaction between the COX, LOX, and CYP pathways and metabolites are warranted. The present results indicate that inhibition of Cyp2C8 could prevent ω3PUFA and ω6PUFA metabolite-induced retinopathy, as has been substantiated by use and observed performance of the Cyp2C8 inhibitor compounds montelukast and fenofibrate in various assays reflective of a therapeutic impact on retinopathy (among other diseases or disorders), including neovascularization, aortic arch growth and HRMEC tubule formation and migration assays.

### Methods

The Examples described herein were carried out, but not limited to, the following methods.

### Oxygen-Induced Ischemic Retinopathy (OIR); PUFA Diet Interventions; Aortic Ring Assay

Mouse model of OIR is described¹³. C57BL/6J mice were analyzed by immunohistochemistry, real-time PCR, western blot, blood smear and LC/MS/MS oxylipid analysis. Mothers of Cyp and sEH mutant mice were fed ω3PUFA or ω6PUFA-enriched diets in OIR followed by analysis of retina, plasma and aortic ring sprouting.

### Animals

All studies adhered to the Association for Research in Vision and Ophthalmology (ARVO) Statement for the Use of Animals in Ophthalmic and Vision Research and were approved by the Children's Hospital Boston Animal Care and Use Committee. Endothelial and circulating cell specific CYP2C8 (Tie2 promoter driven) overexpressing transgenic mice (Tie2-CYP2C8Tg), Endothelial and circulating cell specific sEH (Tie2 promoter driven) overexpressing transgenic mice (Tie2-sEHTg), systemic sEH knockout mice (sEH−/−) were gifts from Dr. Darryl C. Zeldin; (NIH/NIEHS) and wild-type control C57Bl/6J mice (stock no. 000664; Jackson Laboratory) were used in this study. The weight of Tie2-CYP2C8Tg was 6.65±0.17 g (Mean±SEM) and the weight of wild-type littermate controls was 6.50±0.05 g. The weight of Tie2-sEHTg was 6.85±0.62 g and the weight of wild-type littermate controls was 6.10±0.61 g. The weight of sEH−/− was 6.50±0.19 g and the weight of wild-type littermate controls was 6.85±0.15 g.

### Oxygen-Induced Retinopathy

The mouse model of oxygen-induced retinopathy has been previously described (Smith et al. Invest Ophthalmol Vis Sci 35: 101-111). To induce vessel loss, mice were exposed to 75% oxygen from postnatal day 7 (P7) to P12. The central retinal vessel obliteration induced by hyperoxic exposure will trigger an excessive angiogenic response that causes neovascularization. Mice were given lethal doses of Avertin (Sigma) intraperitoneally at P17 when the neovascular response is greatest.

### Immunohistochemistry

Enucleated eyes from wild-type normoxic and hyperoxic P17 mice were fixed for 1 hour at room temperature in 4% paraformaldehyde. For whole mount immunostaining, retinas were dissected, permeabilized for 2 hours at room temperature with 1% Triton X-100 (Sigma, Cat. T-8787) in PBS, and stained with rabbit anti-mouse CYP2C (Abcam, Cat. ab22596, 1:100 dilution), rat anti-mouse F4/80 (Abcam, Cat. ab6640, 1:100 dilution) and Isolectin B4 to visualize vessels, as described above. For retinal cross-section immunostaining, the lens was removed after 1-hour fixation. Eye cups were incubated in 30% sucrose at 4° C. and embedded in Optimal Cutting Tissue medium (OCT). 10 μm-thick sections were cut onto VistaVision Histobond Adhesive Slides (VWR, Cat. 16004-406) and blocked in PBS with 0.1% Triton X-100 and 5% goat serum. Sections were stained with Isolectin B4 and primary antibody goat anti-mouse sEH (Santa Cruz, Cat. sc-22344, 1:200 dilution) followed by secondary antibodies. Retinas were visualized with a Leica SP2 confocal microscope using a 40× objective with 2× zoom. For wholemounts, a stack of optical sections was taken at intervals of 0.16 microns and compiled to reconstruct a 3-dimensional image in the YZ plane using Velocity software.

RNA Isolation and cDNA Preparation

Total RNA was extracted from the retinas of 6 mice each from a different litter at several time points; the RNA was pooled to reduce biologic variability (n=6). Retinas from each time point were lysed with a mortar and pestle and filtered through QiaShredder columns (Qiagen, Cat. 79656). RNA was then extracted as per manufacturer's instructions using the RNeasy Kit (Qiagen, Cat. 74104). To generate cDNA, 1 μg total RNA was treated with DNase I (Qiagen, Cat. 79254) to remove any contaminating genomic DNA, and was then reverse transcribed using random hexamers, and SuperScript III reverse transcriptase (Life Technologies Corp., Cat. 18080-044). All cDNA samples were aliquoted and stored at −80° C.

### Real-Time Polymerase Chain Reaction

PCR primers targeting Cyp2c55 (F: 5′-AATGA TCTGGGGGTGATTTTCAG-3′, R: 5′-GCGATCCTCGATGCTCCTC-3′), sEH (F: 5′-ATCTGAAGCCAGCCCGTGAC-3′, R: 5′-CTGGGCCAGAGCAGGGATCT-3′) and an unchanging control gene cyclophilin A (F: 5′-AGGTGGAGAGCACCAAGACAGA-3′, R: 5′-TGCCGGAGTCGACAATGAT-3′) were designed using Harvard Primer Bank and NCBI Primer Blast Software. Quantitative analysis of gene expression was generated using an ABI Prism 7700 Sequence Detection System with the SYBR Green Master mix kit (Kapa BioSystems, Cat. KK4602). Gene expression was calculated relative to cyclophilin A using the ΔcT method.

### Western Blot Protein Analysis

Normoxic and hyperoxic wild-type mice were sacrificed at postnatal day (P) 9, 12, 14 and 17. Retinas were collected, homogenized and sonicated in cell lysis buffer (Cell Signalling, Cat. 9803) with protease inhibitor (1:1000 dilution). Samples were normalized using a Pierce™ BCA Protein Assay Kit (ThermoScientific, Cat. 23255). 50 μg of retinal lysate were loaded on an SDS-PAGE gel separated by their molecular weights and transferred onto a PVDF membrane. After blocking, the membranes were incubated overnight with primary antibodies goat anti-mouse sEH (Santa Cruz, Cat. sc-22344) or rabbit anti-mouse CYP2C (Abcam, Cat. ab22596) in 5% BSA at 4° C. Secondary incubations with horseradish peroxidase-conjugated rabbit anti-goat and donkey anti-rabbit IgGs (1:10000 dilution) followed for 1 hour at room temperature. Chemiluminescence signals were generated with ECL plus substrate and captured with KODAK film. Densitometry was analysed using ImageJ 1.46r (NIH) software.

### Dietary Intervention

For dietary experiments, polyunsaturated fatty acids (PUFAs), arachidonic acid (AA) and docosahexaenoic acid (DHA) supplements under the trade names ROPUFA, ARASCO and DHASCO, respectively, were obtained from DSM Nutritional Products (dsmnutritionalproducts.com) and were integrated into the rodent feed at Research Diets Incorporated (researchdiets.com/). Diets were stable over time and with oxygen exposure. Upon delivery, dams were fed a defined rodent diet with 10% (w/w) safflower oil containing either 2% ω-6 PUFAs (AA) and no ω-3 PUFAs (DHA and EPA), or 2% ω-3 PUFAs and no ω-6 PUFAs.

### Quantification of Retinal Vaso-Obliteration and Neovascularization

OIR eyes were enucleated and fixed in 4% paraformaldehyde for 1 hour at 4° C. Retinas were dissected and stained overnight at 23° C. with Alexa Fluor 594 fluoresceinated Griffonia Bandereiraea Simplicifolia Isolectin B4 (Molecular Probes, Cat. 121413, 1:100 dilution) in 1 mM CaCl₂ in PBS. Following 2 hours of washes, retinas were whole-mounted onto Superfrost/Plus microscope slides (Fisher, Cat. 12-550-15) with the photoreceptor side up and embedded in SlowFade Antifade reagent (Invitrogen, Cat. S2828). For quantification of retinal neovascularization, 20 images of each whole-mounted retina were obtained at 5× magnifications on a Zeiss AxioObserver.Z1 microscope and merged to form one image with AxioVision 4.6.3.0 software. Vaso-obliteration was quantified using Adobe Photoshop and neovascularization was analysed with the SWIFT_NV method on ImageJ 1.46r (NIH) software, as described previously (Stahl et al. Angiogenesis 12: 297-301).

Macrovascular Sprouting from Ex Vivo Aortic Ring Explants

Tie2-CYP2C8Tg, Tie2-sEHTg, sEH−/− mice and littermate wild-type mice were anesthetized and perfused intracardiacly with warm PBS. Aortae were dissected free, cut into 1-mm-thick rings and embedded in 304, of growth factor-reduced Matrigel™ (BD Biosciences, Cat. 354230) in 24-well tissue culture plates. 5004, CSC complete medium (Cell Systems, Cat. 420-500) activated with growth factor Boost, was then added to each well and incubated at 37° C. with 5% CO₂ for 48 hours before any treatment. Medium contained 5 units/mL of Penicillin/Streptomycin (GIBCO, Cat. 15142) to prevent contamination.

DHA (Cayman Chemical, Cat. 90310, 30 μM) and AA (Cayman Chemical, Cat. 90010, 30 μM) were introduced to the culture medium 48 hours after seeding of aortic ring from Tie2-CYP2C8Tg and littermate wild-type control. 17(18)-EpETE (EEQ) (Cayman Chemical, Cat. 50861, 1 μM), 19(20)-EpDPE (EDP) (Cayman Chemical, Cat. 10175, 1 μM) and 14,15-EE-8(Z)-E (EET) (Cayman Chemical, Cat. 10010486, 1 μM) were administered to the culture medium 48 hours after seeding of aortic ring from Tie2-sEHTg, sEH−/− and their wild-type littermate controls. Medium was changed every 48 hours for all groups. Phase contrast photos of individual explants were taken 168 hours after plating (120 hours after treatment) using a ZEISS Axio Oberver.Z1 microscope. The areas of macrovascular sprouting were quantified with computer software ImageJ 1.46r (National Institute of Health). A semi-automated macro plugin for quantification of vessel sprouts is available from the authors.

### Statistical Analysis

Data are presented as mean±SEM for all histograms unless otherwise indicated. Since the samples were normally distributed, comparisons between groups were made by either 2-tail unpaired student's T-test or analysis of variance (ANOVA) followed by post-hoc Bonferroni correction for comparison among means. P<0.05 is considered statistically significant.

### INCORPORATION BY REFERENCE

All patents, published patent applications and other references disclosed herein are hereby expressly incorporated by reference in their entireties by reference.

### EQUIVALENTS

Those skilled in the art will recognize, or be able to ascertain using no more than routine experimentation, many equivalents of the specific embodiments of the invention described herein. Such equivalents are intended to be encompassed by the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

### REFERENCES

1. Hellstrom A, Smith L E, Dammann O Retinopathy of prematurity. Lancet. Jun. 14, 2013.
2. Connor K M, SanGiovanni J P, Lofqvist C, Aderman C M, Chen J, Higuchi A, Hong S, Pravda E A, Majchrzak S, Carper D, Hellstrom A, Kang J X, Chew E Y, Salem N, Jr., Serhan C N, Smith L E. Increased dietary intake of omega-3-polyunsaturated fatty acids reduces pathological retinal angiogenesis. Nat Med. July 2007; 13(7):868-873.
3. Sapieha P, Stahl A, Chen J, Seaward M R, Willett K L, Krah N M, Dennison R J, Connor K M, Aderman C M, Liclican E, Carughi A, Perelman D, Kanaoka Y, Sangiovanni J P, Gronert K, Smith L E. 5-Lipoxygenase metabolite 4-HDHA is a mediator of the antiangiogenic effect of omega-3 polyunsaturated fatty acids. Science translational medicine. Feb. 9, 2011; 3(69):69ra12.
4. Arnold C, Konkel A, Fischer R, Schunck W H. Cytochrome P450-dependent metabolism of omega-6 and omega-3 long-chain polyunsaturated fatty acids. Pharmacol Rep. May-June 2010; 62(3):536-547.
5. Panigrahy D, Greene E R, Pozzi A, Wang D W, Zeldin D C. EET signaling in cancer. Cancer metastasis reviews. December 2011; 30(3-4):525-540.
6. Michaelis U R, Fisslthaler B, Barbosa-Sicard E, Falck J R, Fleming I, Busse R. Cytochrome P450 epoxygenases 2C8 and 2C9 are implicated in hypoxia-induced endothelial cell migration and angiogenesis. Journal of cell science. Dec. 1, 2005; 118(Pt 23):5489-5498.
7. Edin M L, Wang Z, Bradbury J A, Graves J P, Lih F B, DeGraff L M, Foley J F, Torphy R, Ronnekleiv O K, Tomer K B, Lee C R, Zeldin D C. Endothelial expression of human cytochrome P450 epoxygenase CYP2C8 increases susceptibility to ischemia-reperfusion injury in isolated mouse heart. FASEB J. October 2011; 25(10):3436-3447.
8. Imig J D, Hammock B D. Soluble epoxide hydrolase as a therapeutic target for cardiovascular diseases. Nat Rev Drug Discov. October 2009; 8(10):794-805.
9. Zhang W, Koerner I P, Noppens R, Grafe M, Tsai H J, Morisseau C, Luria A, Hammock B D, Falck J R, Alkayed N J. Soluble epoxide hydrolase: a novel therapeutic target in stroke. Journal of cerebral blood flow and metabolism: official journal of the International Society of Cerebral Blood Flow and Metabolism. December 2007; 27(12):1931-1940.
10. Imig J D. Epoxides and soluble epoxide hydrolase in cardiovascular physiology. Physiol Rev. January 2012; 92(1):101-130.
11. Konkel A, Schunck W H. Role of cytochrome P450 enzymes in the bioactivation of polyunsaturated fatty acids. Biochim Biophys Acta. January 2011; 1814(1):210-222.
12. Zhang G, Panigrahy D, Mahakian L M, Yang J, Liu J Y, Stephen Lee K S, Wettersten H I, Ulu A, Hu X, Tam S, Hwang S H, Ingham E S, Kieran M W, Weiss R H, Ferrara K W, Hammock B D. Epoxy metabolites of docosahexaenoic acid (DHA) inhibit angiogenesis, tumor growth, and metastasis. Proc Natl Acad Sci USA. Apr. 3, 2013.
13. Smith L E, Wesolowski E, McLellan A, Kostyk S K, D'Amato R, Sullivan R, D'Amore P A. Oxygen-induced retinopathy in the mouse. Invest Ophthalmol Vis Sci. January 1994; 35(1):101-111.
14. Michaelis U R, Falck J R, Schmidt R, Busse R, Fleming I. Cytochrome P4502C9-derived epoxyeicosatrienoic acids induce the expression of cyclooxygenase-2 in endothelial cells. Arterioscler Thromb Vasc Biol. February 2005; 25(2):321-326.
15. Revermann M, Mieth A, Popescu L, Paulke A, Wurglics M, Pellowska M, Fischer A S, Steri R, Maier T J, Schermuly R T, Geisslinger G, Schubert-Zsilavecz M, Brandes R P, Steinhilber D. A pirinixic acid derivative (LP105) inhibits murine 5-lipoxygenase activity and attenuates vascular remodelling in a murine model of aortic aneurysm. Br J Pharmacol. August 2011; 163(8):1721-1732.
16. Liu X, Zhu P, Freedman B D. Multiple eicosanoid-activated nonselective cation channels regulate B-lymphocyte adhesion to integrin ligands. Am J Physiol Cell Physiol. March 2006; 290(3):C873-882.

1. A method of treating or preventing vascular diseases of the retina in a subject, comprising administering to a subject a therapeutically effective amount of an inhibitor of cytochrome P450 2C8 (CYP2C8) activity or expression, thereby treating or preventing vascular diseases of the retina.

2. A method selected from the group consisting of:
a method of treating or preventing angiogenesis in a subject, comprising administering to a subject a therapeutically effective amount of an inhibitor of CYP2C8 activity or expression, thereby treating or preventing angiogenesis;
a method of treating or preventing neovascularization in a subject, comprising administering to a subject a therapeutically effective amount of an inhibitor of CYP2C8 activity or expression, thereby treating or preventing neovascularization;
a method of treating or preventing vascular diseases of the retina in a subject, comprising administering to a subject a therapeutically effective amount of a promoter of soluble epoxide hydrolase (sEH) activity or expression, thereby treating or preventing vascular diseases of the retina;
a method of treating or preventing a vascular disease of the retina, angiogenesis and/or neovascularization in a subject, comprising administering to a subject a therapeutically effective amount of montelukast or fenofibrate, thereby treating or preventing a vascular disease of the retina, angiogenesis and/or neovascularization in the subject;
a method of treating or preventing angiogenesis in a subject, comprising administering to a subject a therapeutically effective amount of a promoter of sEH activity or expression, thereby treating or preventing angiogenesis; and
a method of treating or preventing neovascularization in a subject, comprising administering to a subject a therapeutically effective amount of a promoter of sEH activity or expression, thereby treating or preventing neovascularization.

3-5. (canceled)

6. The method of claim 1, wherein the vascular diseases of the retina are selected from the group consisting of: retinopathy, exudative age related macular degeneration (ARMD), and vascular occlusions.

7. The method of claim 6, wherein the retinopathy is selected from diabetic retinopathy and retinopathy of prematurity (ROP).

8-9. (canceled)

10. The method of claim 1, wherein the subject is identified as having a vascular disease of the retina or as being predisposed to having a vascular disease of the retina.

11. The method of claim 10, wherein the vascular diseases of the retina are selected from the group consisting of: retinopathy, exudative age related macular degeneration (ARMD), and vascular occlusions.

12. The method of claim 1, wherein the subject is a prematurely delivered infant at risk for retinopathy of prematurity.

13. The method of claim 1, wherein montelukast, fenofibrate and/or the inhibitor of CYP2C8 decreases the activity of a CYP2C8 protein or decreases the expression of a CYP2C8 gene in the tissue.

14. The method of claim 1, wherein the promoter of sEH increases the activity of a sEH protein or increases the expression of a sEH gene in the tissue.

15. The method of claim 1, wherein montelukast, fenofibrate, the inhibitor of CYP2C8 activity and/or promoter of sEH activity or expression is administered to ocular tissue.

16. The method of claim 1, wherein the retinopathy is selected from the group consisting of diabetic retinopathy, retinopathy of prematurity, and wet age-related macular degeneration.

17. The method of claim 1, wherein the subject is being fed a polyunsaturated fatty acid (PUFA) enriched diet.

18. The method of claim 17, wherein the PUFA enriched diet is enriched in ω3-PUFA or ω-6 PUFA.

19. The method of claim 1, further comprising administering an inhibitor of CYP2J2 to the subject.

20. The method of claim 19, wherein the inhibitor of CYP2J2 is selected from the group consisting of Telmisartan, Flunarizine, Amodiaquine, Nicardipine, Mibefradil, Norfloxacin, Nifedipine, Nimodipine, Benzbromarone and Haloperidol.

21. A pharmaceutical composition for treatment of a vascular disease of the retina in a subject comprising montelukast or fenofibrate and instructions for its use.


SEQUENCE LISTING (6 sequences)

SEQ ID NO: 1 - DNA, 23 bp, Artificial Sequence
  Cyp2c55 Forward
aatgatctgggggtgattttcag

SEQ ID NO: 2 - DNA, 19 bp, Artificial Sequence
  2. Cyp2c55 Reverse
gcgatcctcgatgctcctc

SEQ ID NO: 3 - DNA, 20 bp, Artificial Sequence
  sEH Forward
atctgaagccagcccgtgac

SEQ ID NO: 4 - DNA, 20 bp, Artificial Sequence
  sEH Reverse
ctgggccagagcagggatct

SEQ ID NO: 5 - DNA, 22 bp, Artificial Sequence
  cyclophilin A Forward
aggtggagagcaccaagacaga

SEQ ID NO: 6 - DNA, 19 bp, Artificial Sequence
  cyclophilin A Reverse
tgccggagtcgacaatgat